(19) United States
(12) Patent Application Publication
(10) Pub. No.: US 2022/0178940 A1
(43) Pub. Date: Jun. 9, 2022

(54) Reducing Intersample Analyte Variability in Complex Biological Matrices

(71) Applicant: SomaLogic, Inc., Boulder, CO (US)

(72) Inventors: Daniel Drolet, Boulder, CO (US); David Astling, Denver, CO (US); Ira von Carlowitz, Broomfield, CO (US); Thomas Howard Hraha, Boulder, CO (US); Jan Hoh, Boulder, CO (US)

(73) Assignee: SomaLogic, Inc., Boulder, CO (US)

(21) Appl. No.: 17/438,120

(22) PCT Filed: Mar. 20, 2020
(86) PCT No.: PCT/US2020/023866

§ 371 Date: Sep. 10, 2021

Related U.S. Application Data
(60) Provisional application No. 62/822,349, filed on Mar. 22, 2019.

(51) Int. Cl. G01N 33/68; G01N 1/34

(52) U.S. Cl. CPC G01N 33/6827; G01N 2015/0693; G01N 1/34

(57) ABSTRACT

Described herein are compositions and methods for reducing the variability of inter-sample analyte measurements from a biological matrix. In some embodiments, the present disclosure relates to methods for reducing the variability in the inter-sample levels of one or more proteins from a biological sample as measured by a proteomic assay.

### FIELD

The present disclosure relates generally to reducing variability of inter-sample analyte measurements from a biological sample. In some embodiments, the present disclosure relates to compositions and methods for reducing the variability in the inter-sample levels of one or more proteins as measured by a proteomic assay.

### BACKGROUND

Inter-sample variability of analyte measurement in biological samples is a problem for biomarker discovery, metabolic analyses, gene expression analysis, protein pathway analysis, and diagnostic and prognostic tools, particularly when the outcome relies on quantitative biological signals that differ by a relatively small magnitude. Biological sample types that exhibit high between-sample variability are a primary challenge of working with these sample types. Reducing such variability would provide for more consistent and meaningful datasets for experimental and clinical applications.

Therefore, there continues to be a need for alternative compositions and methods for reducing inter-sample analyte measurement variability in biological matrices. The present disclosure meets such needs by providing novel compositions and methods for normalizing biological signals in complex matrices, which reduce, minimize, or remove such variability.

### SUMMARY

Provided herein are buffer-exchanged biological samples and methods of preparing biological samples for detecting a protein comprising buffer exchange. In some embodiments, there is reduced inter-sample variability of analyte levels measured in test samples comprising or generated from the buffer-exchanged biological samples relative to samples comprising or generated from non-buffer-exchanged biological samples. In some embodiments, there is reduced inter-sample variability of analyte levels in test samples generated using the methods comprising buffer exchange relative to methods that do not comprise buffer exchange. In some embodiments, the linear range of measurement for analytes in the test samples comprising or generated from buffer-exchanged biological samples is greater than the linear range of measurement for analytes in test samples comprising or generated from non-buffer-exchanged biological samples. In some embodiments, the analyte is a protein, such as a target protein. In some embodiments, the biological sample is a urine sample. In some embodiments, the total protein concentration in the test sample is from 2 μg/mL to 100 μg/mL. In some embodiments, the total protein concentration in the test sample is from 2 μg/mL to 60 μg/mL. In some such embodiments, the total protein concentration of the test sample or of the biological sample is adjusted to the range of from about 2 μg/mL to about 60 μg/mL following determination that the total protein concentration is not in the range. In some embodiments, the total protein concentration of the test sample is in the range of from about 2 μg/mL to about 60 μg/mL without an adjustment of total protein concentration. In some such embodiments, the total protein concentration is measured using a fluorescence readout assay. In some embodiments, the total protein concentration in the test sample is from 70 μg/mL to 100 μg/mL. In some such embodiments, the total protein concentration of the test sample is adjusted to the range of from about 70 μg/mL to about 100 μg/mL following determination that the total protein concentration is not in the range. In some embodiments, the total protein concentration of the test sample is in the range of from about 70 μg/mL to about 100 μg/mL without an adjustment of total protein concentration. In some such embodiments, the total protein concentration is measured using a BCA assay. In some embodiments, the total protein concentration of each of a plurality of test samples or of a plurality of biological samples is adjusted to be the same.

Certain embodiments provided herein are listed below.
Embodiment 1. A method for preparing a biological sample for detecting a protein comprising:

a) generating a test sample by performing a buffer exchange on the biological sample using a formulation comprising a buffering agent, one or more salts, a chelating agent and a nonionic surfactant; and

b) generating an adjusted test sample by adjusting the total protein concentration of the test sample, wherein the total protein concentration in the adjusted test sample is from about 2 μg/mL to about 60 μg/mL.

Embodiment 2. The method of embodiment 1 comprising determining the protein concentration of the test sample.
Embodiment 3. A method for preparing a biological sample for detecting a protein comprising:

a) generating a test sample by performing a buffer exchange on the biological sample using a formulation comprising a buffering agent, one or more salts, a chelating agent and a nonionic surfactant;

b) determining the total protein concentration of the test sample; and

c) generating an adjusted test sample by adjusting the total protein concentration of the test sample, wherein the total protein concentration in the adjusted test sample is from about 2 μg/mL to about 60 μg/mL.

Embodiment 4. The method of any one of embodiments 1-3, comprising generating a plurality of test samples by performing a buffer exchange on a plurality of biological samples, wherein the total protein concentration of each adjusted test sample is about the same.
Embodiment 5. The method of any one of embodiments 1-3, comprising generating a plurality of test samples by performing a buffer exchange on a plurality of biological samples, wherein the total protein concentration of each adjusted test sample is the same.
Embodiment 6. A method of preparing a biological sample for detecting a protein comprising:

a) generating a test sample by performing a buffer exchange on the biological sample using a formulation comprising a buffering agent, one or more salts, a chelating agent and a nonionic surfactant;

b) determining the protein concentration of the test sample; and

c) if the total protein concentration of the test sample is not in the range of from about 2 μg/mL to about 60 μg/mL, generating an adjusted test sample by adjusting the total protein concentration of the test sample, wherein the total protein concentration in the adjusted test sample is from about 2 μg/mL to about 60 μg/mL.

Embodiment 7. The method of embodiment 6, wherein if the total protein concentration of the test sample is not in the range of from about 2 μg/mL to about 50 μg/mL; or from about 2 μg/mL to about 45 μg/mL; or from about 4 μg/mL to about 40 μg/mL; or from about 8 μg/mL to about 40 μg/mL; or from about 10 μg/mL to about 40 μg/mL; or from about 10 μg/mL to about 30 μg/mL; or less than 40 μg/mL; or less than 30 μg/mL; or less than 20 μg/mL; or about 15 μg/mL; or from about 10 μg/mL to about 20 μg/mL, generating an adjusted test sample by adjusting the total protein concentration of the test sample to the range.
Embodiment 8. The method of any one of embodiments 1-7, wherein the one or more salts are each independently selected from a sodium salt, a potassium salt and a magnesium salt. Embodiment 9. The method of any one of embodiments 1-7, wherein the one or more salts comprise a sodium salt, a potassium salt and a magnesium salt.
Embodiment 10. The method of any one of embodiments 8 or 9, wherein the sodium salt is NaCl, the potassium salt is KCl and the magnesium salt is MgCl₂.
Embodiment 11. The method of embodiment 10, wherein the NaCl in the formulation is at a concentration of from about 10 mM to about 500 mM, or from about 50 mM to about 250 mM, or from about 100 mM to about 200 mM, or about 102 mM.
Embodiment 12. The method of embodiment 10 or 11, wherein the KCl in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 13. The method of any one of embodiments 10-12, wherein the MgCl₂ in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 14. The method of any one of embodiments 1-13, wherein the buffering agent is selected from HEPES, MES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.
Embodiment 15. The method of embodiment 14, wherein the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 50 mM, or about 40 mM.
Embodiment 16. The method of any one of embodiments 1-15, wherein the chelating agent is selected from EDTA, EGTA, DTPA, BAPTA, DMPS and ALA.
Embodiment 17. The method of embodiment 16, wherein the chelating agent in the formulation is at a concentration of about 0.1 mM to about 10 mM, or from about 0.5 mM to about 5 mM, or about 1 mM.
Embodiment 18. The method of any one of embodiments 1-17, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).
Embodiment 19. The method of embodiment 18, wherein the nonionic surfactant is from about 0.01% to about 1% of the formulation, or about 0.02% to about 0.5% of the formulation, or from about 0.03% to about 0.1% of the formulation, or from about 0.04% to about 0.08% of the formulation, or about 0.05%, or about 0.1% of the formulation, volume for volume.
Embodiment 20. The method of any one of embodiments 1-19, wherein the total protein concentration of the adjusted test sample is from about 2 μg/mL to about 50 μg/mL; or from about 2 μg/mL to about 45 μg/mL; or from about 4 μg/mL to about 40 μg/mL; or from about 8 μg/mL to about 40 μg/mL; or from about 10 μg/mL to about 40 μg/mL; or from about 10 μg/mL to about 30 μg/mL; or less than 40 μg/mL; or less than 30 μg/mL; or less than 20 μg/mL; or about 15 μg/mL; or from about 10 μg/mL to about 20 μg/mL.
Embodiment 21. The method of any one of embodiments 2-20, wherein the total protein concentration of the test sample or the adjusted test sample is determined with an assay selected from the group consisting of a fluorescence readout assay, a Bradford assay, a bicinchoninic acid (BCA) assay, a Lowry assay and an ELISA assay.
Embodiment 22. The method of embodiment 21, wherein the fluorescence readout assay comprises a reagent selected from a merocyanine dye and epicocconone.
Embodiment 23. The method of any one of embodiments 1-22, wherein the formulation comprises 40 mM HEPES, 102 mM NaCl, 5 mM KCl, 5 mM MgCl₂, 1 mM EDTA and 0.05% Tween-20.
Embodiment 24. The method of embodiment any one of embodiments 1-23, wherein the pH of the formulation is from about pH 5 to about pH 9, or from about pH 6 to about pH 8, or from about pH 7 to about pH 7.9, or about pH 7.5.
Embodiment 25. The method of any one of embodiments 1-24, wherein the formulation consists of 40 mM HEPES, 102 mM NaCl, 5 mM KCl, 5 mM MgCl₂, 1 mM EDTA and 0.05% Tween-20 and has a pH of 7.5.
Embodiment 26. The method of any one of embodiments 1-25, wherein the buffer exchange is not performed with ultrafiltration.
Embodiment 27. The method of any one of embodiments 1-26, wherein the buffer exchange is performed with gel filtration chromatography.
Embodiment 28. The method of any one of embodiments 1-27, wherein the biological sample is a urine sample.
Embodiment 29. The method of any one of embodiments 1-27, wherein the biological sample is a serum sample.
Embodiment 30. The method of any one of embodiments 1-29, wherein the method does not comprise a concentrating the biological sample.
Embodiment 31. A test sample comprising a buffer-exchanged biological sample, wherein the test sample has a total protein concentration from about 2 μg/mL to about 60 μg/mL, and wherein the buffer-exchanged biological sample comprises a buffering agent, one or more salts, a chelating agent and a nonionic surfactant.
Embodiment 32. The test sample of embodiment 31, further comprising one or more protein capture reagents.
Embodiment 33. The test sample of embodiment 32, wherein each of the one or more protein capture reagents is an aptamer or an antibody.
Embodiment 34. The test sample of any one of embodiments 31-33, wherein the total protein concentration of the test sample is from about 2 μg/mL to 50 μg/mL; or from about 2 μg/mL to 45 μg/mL; or from about 4 μg/mL to 40 μg/mL; or from about 8 μg/mL to 40 μg/mL; or from about 10 μg/mL to 40 μg/mL; or from about 10 μg/mL to 30 μg/mL; or is less than 50 μg/mL; or is less than 40 μg/mL; or is less than 30 μg/mL; or is less than 20 μg/mL; or is about 15 μg/mL; or is from about 10 μg/mL to about 20 μg/mL.
Embodiment 35. The test sample of any one of embodiments 31-34, wherein the one or more salts are each independently selected from a sodium salt, a potassium salt and a magnesium salt.
Embodiment 36. The test sample of any one of embodiments 31-34, wherein the one or more salts comprise a sodium salt, a potassium salt and a magnesium salt.
Embodiment 37. The test sample of embodiment 35 or 36, wherein the sodium salt is NaCl, the potassium salt is KCl and the magnesium salt is MgCl₂.
Embodiment 38. The test sample of embodiment 37, wherein the NaCl in the test sample is at a concentration of from about 10 mM to about 500 mM, or from about 50 mM to about 250 mM, or from about 100 mM to about 200 mM or about 102 mM.
Embodiment 39. The test sample of embodiment 37 or 38, wherein the KCl in the test sample is at a concentration of from about 0.5 mM to about 30 mM, or from about 1 mM to about 20 mM, or from about 2 mM to about 15 mM, or from about 4 mM to about 10 mM, or about 5 mM.
Embodiment 40. The test sample of any one of embodiments 37-39, wherein the MgCl₂ in the test sample is at a concentration of from about 0.5 mM to about 30 mM, or from about 1 mM to about 20 mM, or from about 2 mM to about 15 mM, or from about 4 mM to about 10 mM, or about 5 mM.
Embodiment 41. The test sample of any one of embodiments 31-40, wherein the buffering agent is selected from HEPES, MES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.
Embodiment 42. The test sample of any one of embodiments 31-41, wherein the buffering agent in the test sample is at a concentration of from about 4 mM to about 400 mM, or from about 10 mM to about 300 mM, or from about 20 mM to about 200 mM, or from about 30 mM to about 100 mM, or from 35 mM to about 50 mM or about 40 mM.
Embodiment 43. The test sample of any one of embodiments 31-42, wherein the chelating agent is selected from EDTA, EGTA, DTPA, BAPTA, DMPS and ALA.
Embodiment 44. The test sample of any one of embodiments 31-43, wherein the chelating agent in the test sample is at a concentration of about 0.1 mM to about 10 mM, or from about 0.5 mM to about 5 mM, or about 1 mM.
Embodiment 45. The test sample of any one of embodiments 31-44, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).
Embodiment 46. The test sample of any one of embodiments 31-45, wherein the nonionic surfactant is from about 0.01% to about 1% of the test sample, or about 0.02% to about 0.5% of the test sample, or from about 0.03% to about 0.1% of the test sample, or from about 0.04% to about 0.08% of the test sample, or about 0.05% of the test sample, volume by volume.
Embodiment 47. The test sample of any one of embodiments 31-46, wherein the total protein concentration of the test sample is determined with an assay selected from the group consisting of a fluorescence readout assay, a Bradford assay, a bicinchoninic acid (BCA) assay, a Lowry assay and an ELISA assay.
Embodiment 48. The test sample of embodiment 47, wherein the fluorescence readout assay comprises a reagent selected from a merocyanine dye and epicocconone.
Embodiment 49. The test sample of any one of embodiments 31-48, wherein the test sample comprises 40 mM HEPES, 102 mM NaCl, 5 mM KCl, 5 mM MgCl₂, 1 mM EDTA and 0.05% Tween 20.
Embodiment 50. The test sample of any one of embodiments 31-49, wherein the pH of the test sample is from about pH 5 to about pH 9, or from about pH 6 to about pH 8, or from about pH 7 to about pH 7.9, or about pH 7.5.
Embodiment 51. The test sample of any one of embodiments 31-50, wherein at least one capture reagent is an aptamer, and wherein the at least one aptamer comprises a 5-position modified pyrimidine.
Embodiment 52. The test sample of embodiment 51, wherein the 5-position modified pyrimidine comprises a hydrophobic moiety at the 5-position of the pyrimidine.
Embodiment 53. The test sample of embodiment 52, wherein the hydrophobic moiety is selected from a naphthyl moiety, a phenyl moiety, a tyrosyl moiety, an indole moiety and a morpholino moiety.
Embodiment 54. The test sample of embodiment 52 or 53, wherein the hydrophobic moiety is covalently linked to the 5-position of the based via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.
Embodiment 55. The test sample of any one of embodiments 31-54, wherein the biological sample is a urine sample.
Embodiment 56. The test sample of any one of embodiments 31-54, wherein the biological sample is a serum sample.
Embodiment 57. A method for detecting a target protein in a test sample comprising:

a) contacting the test sample with at least one capture reagent, wherein the at least one capture reagent is capable of binding to the target protein to form a complex;

b) incubating the test sample with the at least one capture reagent under conditions that allow for the complex to form; and

c) determining the level of the target protein in the test sample by measuring the level of the at least one capture reagent, the complex, or the protein; wherein the level of the at least one capture reagent or the complex is a surrogate for the level of the target protein;

wherein, the test sample is generated by performing a buffer exchange of a biological sample with a formulation comprising a buffering agent, a salt, a chelating agent and a nonionic surfactant; and

wherein, the total protein concentration of the test sample is about 2 μg/mL to about less than 60 μg/mL.

Embodiment 58. The method of embodiment 57, wherein the at least one protein capture reagent is an aptamer or an antibody.
Embodiment 59. The method of embodiment 57 or 58 comprising a plurality of capture reagents, where each capture reagent is an aptamer.
Embodiment 60. The method of any one of embodiments 57-59, wherein the total protein concentration of the test sample is about 2 μg/mL to 50 μg/mL; or about 2 μg/mL to 45 μg/mL; or about 4 μg/mL to 40 μg/mL; or about 8 μg/mL to 40 μg/mL; or about 10 μg/mL to 40 μg/mL; or about 10 μg/mL to 30 μg/mL; or less than 50 μg/mL; or less than 40 μg/mL; or less than 30 μg/mL; or less than 20 μg/mL; or about 15 μg/mL; or about 10 μg/mL to about 20 μg/mL.
Embodiment 61. The method of any one of embodiments 57-60, wherein the one or more salts are selected from a sodium salt, a potassium salt and a magnesium salt.
Embodiment 62. The method of any one of embodiments 57-60, wherein the one or more salts comprise a sodium salt, a potassium salt and a magnesium salt.
Embodiment 63. The method of embodiment 61 or 62, wherein the sodium salt is NaCl, the potassium salt is KCl and the magnesium salt is MgCl₂.
Embodiment 64. The method of embodiment 63, wherein the NaCl in the formulation is at a concentration of from about 10 mM to about 500 mM, or from about 50 mM to about 250 mM, or from about 100 mM to about 200 mM or about 102 mM.
Embodiment 65. The method of embodiment 63 or 64, wherein the KCl in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 66. The method of any one of embodiments 63-65, wherein the MgCl₂ in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 67. The method of any one of embodiments 57-66, wherein the buffering agent is selected from HEPES, MES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.
Embodiment 68. The method of any one of embodiments 57-67, wherein the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 50 mM or about 40 mM.
Embodiment 69. The method of any one of embodiments 57-68, wherein the chelating agent is selected from EDTA, EGTA, DTPA, BAPTA, DMPS and ALA.
Embodiment 70. The method of any one of embodiments 57-69, wherein the chelating agent in the formulation is at a concentration of about 0.1 mM to about 10 mM, or from about 0.5 mM to about 5 mM, or about 1 mM.
Embodiment 71. The method of any one of embodiments 57-70, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).
Embodiment 72. The method of any one of embodiments 57-71, wherein the nonionic surfactant is from about 0.01% to about 1% of the formulation, or about 0.02% to about 0.5% of the formulation, or from about 0.03% to about 0.1% of the formulation, or from about 0.04% to about 0.08% of the formulation, or about 0.05% of the formulation.
Embodiment 73. The method of any one of embodiments 57-72, wherein the total protein concentration of the test sample is measured with an assay selected from the group consisting of a fluorescence readout assay, a Bradford assay, a bicinchoninic acid (BCA) assay, a Lowry assay and an ELISA assay.
Embodiment 74. The method of embodiment 73, wherein the fluorescence readout assay comprises a reagent selected from a merocyanine dye and epicocconone.
Embodiment 75. The method of any one of embodiments 57-74, wherein the formulation comprises 40 mM HEPES, 102 mM NaCl, 5 mM KCl, 5 mM MgCl₂, 1 mM EDTA and 0.05% Tween 20.
Embodiment 76. The method of any one of embodiments 57-74, wherein the formulation consists of 40 mM HEPES, 102 mM NaCl, 5 mM KCl, 5 mM MgCl₂, 1 mM EDTA and 0.05% Tween 20 at pH 7.5.
Embodiment 77. The method of any one of embodiments 57-75, wherein the pH of the formulation is from about pH 5 to about pH 9, or from about pH 6 to about pH 8, or from about pH 7 to about pH 7.9, or about pH 7.5.
Embodiment 78. The method of any one of embodiments 58-77, wherein the aptamer comprises a 5-position modified pyrimidine.
Embodiment 79. The method of embodiment 78, wherein the 5-position modified pyrimidine comprises a hydrophobic moiety at the 5-position of the pyrimidine.
Embodiment 80. The method of embodiment 79, wherein the hydrophobic moiety is selected from a naphthyl moiety, a phenyl moiety, a tyrosyl moiety, an indole moiety and a morpholino moiety.
Embodiment 81. The method of embodiment 79 or 80, wherein the hydrophobic moiety is covalently linked to the 5-position of the based via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.
Embodiment 82. The method of any one of embodiments 57-81, wherein the complex is a non-covalent complex.
Embodiment 83. The method of any one of embodiments 57-82, wherein the at least one capture reagent is attached to a first solid support before contacting the test sample or the capture reagent is attached to a first solid support after contacting the test sample.
Embodiment 84. The method of any one of embodiments 57-83 further comprising attaching the complex to a first solid support via the capture reagent.
Embodiment 85. The method of embodiment 84 further comprising releasing complex from the first solid support and attaching the complex to a second solid support.
Embodiment 86. The method of embodiment 85, wherein the complex is attached to the second solid support via the protein.
Embodiment 87. The method of any one of embodiments 57-86 further comprising adding a competitor molecule to the test sample and/or adding a competitor molecule and diluting the test sample.
Embodiment 88. The method of embodiment 87, wherein the competitor molecule is a polyanionic competitor.
Embodiment 89. The method of embodiment 88, wherein the polyanionic competitor is selected from an oligonucleotide, polydextran, DNA, heparin and dNTPs.
Embodiment 90. The method of embodiment 89, wherein the polyanionic competitor is a polydextran, and wherein the polydextran is dextran sulfate.
Embodiment 91. The method of embodiment 89, wherein the polyanionic competitor is an oligonucleotide, and wherein the oligonucleotide comprises one or more 5-position modified pyrimidines.
Embodiment 92. The method of any one of embodiments 57-91, wherein the biological sample is urine.
Embodiment 93. The method of any one of embodiments 57-91, wherein the biological sample is serum.
Embodiment 94. The method of any one of embodiments 1-30 or 57-93, wherein the buffer exchange is performed using gel filtration chromatography.
Embodiment 95. The test sample of any one of embodiments 31-56, wherein gel filtration chromatography was used to perform the buffer exchange on the biological sample in order to generate the buffer-exchanged biological sample.
Embodiment 96. The method of any one of embodiments 1-30 or 57-94, further comprising measuring the protein concentration of the biological sample prior to the performing of the buffer exchange.
Embodiment 97. The method of embodiment 96, wherein the protein concentration of the biological sample is measured with an assay selected from the group consisting of a fluorescence readout assay, a bicinchoninic acid (BCA) assay, a micro BCA assay, a Lowry assay, and an ELISA assay.
Embodiment 98. The test sample of any one of embodiments 47-56 or 95, wherein the assay used to determine the total protein concentration is performed prior to buffer exchange of the biological sample.
Embodiment 99. The test sample of any one of embodiments 47-56, 95, or 98, wherein the assay used to determine the total protein concentration is performed after buffer exchange of the biological sample.
Embodiment 100. A method for preparing a plurality of biological samples for detecting a protein comprising:

generating a plurality of test samples by performing a buffer exchange on the plurality of biological samples using a formulation comprising a buffering agent, one or more salts, a chelating agent and a nonionic surfactant; and

generating a plurality of adjusted test samples by adjusting the total protein concentrations of a plurality of test samples, wherein the total protein concentration of each adjusted test sample is about the same;

and wherein the method does not comprise concentrating the total protein of the plurality of biological samples prior to performing the buffer exchange.

Embodiment 101. A method for preparing a biological sample for detecting a protein comprising:

generating a test sample by performing a buffer exchange on the biological sample using a formulation comprising a buffering agent, one or more salts, a chelating agent and a nonionic surfactant; and

generating an adjusted test sample by adjusting the total protein concentration of the test sample, wherein the total protein concentration in the adjusted test sample is from about 70 μg/mL to about 100 μg/mL.

Embodiment 102. The method of embodiment 100 or 101 comprising determining the protein concentration of the test sample or plurality of test samples and/or the biological sample or plurality of biological samples.
Embodiment 103. A method for preparing a biological sample for detecting a protein comprising:

generating a test sample by performing a buffer exchange on the biological sample using a formulation comprising a buffering agent, one or more salts, a chelating agent and a nonionic surfactant;

determining the total protein concentration of the test sample; and

generating an adjusted test sample by adjusting the total protein concentration of the test sample, wherein the total protein concentration in the adjusted test sample is from about 70 μg/mL to about 100 μg/mL.

Embodiment 104. The method of any one of embodiments 101-103, comprising generating a plurality of test samples by performing a buffer exchange on a plurality of biological samples, wherein the total protein concentration of each adjusted test sample is about the same.
Embodiment 105. The method of any one of embodiments 100-103, comprising generating a plurality of test samples by performing a buffer exchange on a plurality of biological samples, wherein the total protein concentration of each adjusted test sample is the same.
Embodiment 106. A method of preparing a biological sample for detecting a protein comprising:

generating a test sample by performing a buffer exchange on the biological sample using a formulation comprising a buffering agent, one or more salts, a chelating agent and a nonionic surfactant;

determining the protein concentration of the test sample; and

if the total protein concentration of the test sample is not in the range of from about 70 μg/mL to about 100 μg/mL, generating an adjusted test sample by adjusting the total protein concentration of the test sample, wherein the total protein concentration in the adjusted test sample is from about 70 μg/mL to about 100 μg/mL.

Embodiment 107. The method of embodiment 106, wherein if the total protein concentration of the test sample is not in the range of from about 70 μg/mL to about 100 μg/mL; or from about 70 μg/mL to about 95 μg/mL; or from about 70 μg/mL to about 90 μg/mL; or from about 70 μg/mL to about 85 μg/mL; or from about 70 μg/mL to about 80 μg/mL; or from about 70 μg/mL to about 75 μg/mL, generating an adjusted test sample by adjusting the total protein concentration of the test sample to the range.
Embodiment 108. The method of any one of embodiments 100-107, wherein the one or more salts are each independently selected from a sodium salt, a potassium salt and a magnesium salt.
Embodiment 109. The method of any one of embodiments 100-107, wherein the one or more salts comprise a sodium salt, a potassium salt and a magnesium salt.
Embodiment 110. The method of any one of embodiments 108 or 109, wherein the sodium salt is NaCl, the potassium salt is KCl and the magnesium salt is MgCl₂.
Embodiment 111. The method of embodiment 110, wherein the NaCl in the formulation is at a concentration of from about 10 mM to about 500 mM, or from about 50 mM to about 250 mM, or from about 100 mM to about 200 mM, or from about 75-125 mM, or about 102 mM.
Embodiment 112. The method of embodiment 110 or 111, wherein the KCl in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 113. The method of any one of embodiments 110-112, wherein the MgCl₂ in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 114. The method of any one of embodiments 100-113, wherein the buffering agent is selected from HEPES, MES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.
Embodiment 115. The method of embodiment 114, wherein the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 50 mM, or about 40 mM.
Embodiment 116. The method of any one of embodiments 100-115, wherein the chelating agent is selected from EDTA, EGTA, DTPA, BAPTA, DMPS and ALA.
Embodiment 117. The method of embodiment 116, wherein the chelating agent in the formulation is at a concentration of about 0.1 mM to about 10 mM, or from about 0.5 mM to about 5 mM, or about 1 mM.
Embodiment 118. The method of any one of embodiments 100-117, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).
Embodiment 119. The method of embodiment 118, wherein the nonionic surfactant is from about 0.01% to about 1% of the formulation, or about 0.02% to about 0.5% of the formulation, or from about 0.03% to about 0.1% of the formulation, or from about 0.04% to about 0.08% of the formulation, or about 0.05%, or about 0.1% of the formulation, volume for volume.
Embodiment 120. The method of any one of embodiments 100-119, wherein the total protein concentration of the adjusted test sample is from about 2 μg/mL to about 70 μg/mL; or from about 2 μg/mL to about 75 μg/mL; or from about 4 μg/mL to about 70 μg/mL; or from about 8 μg/mL to about 70 μg/mL; or from about 10 μg/mL to about 70 μg/mL; or from about 10 μg/mL to about 70.
Embodiment 121. The method of any one of embodiments 102-120, wherein the total protein concentration of the test sample or the adjusted test sample is determined with a bicinchoninic acid (BCA) assay, wherein the BCA assay is optionally a micro BCA assay.
Embodiment 122. The method of embodiment 121, wherein the assay comprises a bicinchoninic acid reagent or a bicinchonic acid reagent, and optionally an alkaline tartrate-carbonate buffer and/or a copper sulfate solution.
Embodiment 123. The method of any one of embodiments 100-122, wherein the formulation comprises 40 mM HEPES, 102 mM NaCl, 5 mM KCl, 5 mM MgCl₂, 1 mM EDTA and 0.05% Tween-20.
Embodiment 124. The method of embodiment any one of embodiments 100-123, wherein the pH of the formulation is from about pH 5 to about pH 9, or from about pH 6 to about pH 8, or from about pH 7 to about pH 7.9, or about pH 7.5.
Embodiment 125. The method of any one of embodiments 100-124, wherein the formulation consists of 40 mM HEPES, 102 mM NaCl, 5 mM KCl, 5 mM MgCl₂, 1 mM EDTA and 0.05% Tween-20 and has a pH of 7.5.
Embodiment 126. The method of any one of embodiments 100-125, wherein the buffer exchange is not performed with ultrafiltration.
Embodiment 127. The method of any one of embodiments 100-126, wherein the buffer exchange is performed with gel filtration chromatography.
Embodiment 128. The method of any one of embodiments 100-127, wherein the biological sample is a urine sample.
Embodiment 129. The method of any one of embodiments 100-127, wherein the biological sample is a serum sample.
Embodiment 130. The method of any one of embodiments 101-129, wherein the method does not comprise a concentrating the biological sample.
Embodiment 131. A test sample comprising a buffer-exchanged biological sample, wherein the test sample has a total protein concentration from about 70 μg/mL to about 100 μg/mL, and wherein the buffer-exchanged biological sample comprises a buffering agent, one or more salts, a chelating agent and a nonionic surfactant.
Embodiment 132. The test sample of embodiment 131, further comprising one or more protein capture reagents.
Embodiment 133. The test sample of embodiment 132, wherein each of the one or more protein capture reagents is an aptamer or an antibody.
Embodiment 134. The test sample of any one of embodiments 131-133, wherein the total protein concentration of the test sample is from about 70 μg/mL to 95 μg/mL; or from about 70 μg/mL to 90 μg/mL; or from about 70 μg/mL to 85 μg/mL; or from about 70 μg/mL to 80 μg/mL; or from about 70 μg/mL to 75 μg/mL; or is about 70 μg/mL.
Embodiment 135. The test sample of any one of embodiments 131-134, wherein the one or more salts are each independently selected from a sodium salt, a potassium salt and a magnesium salt.
Embodiment 136. The test sample of any one of embodiments 131-135, wherein the one or more salts comprise a sodium salt, a potassium salt and a magnesium salt.
Embodiment 137. The test sample of embodiment 135 or 136, wherein the sodium salt is NaCl, the potassium salt is KCl and the magnesium salt is MgCl₂.
Embodiment 138. The test sample of embodiment 137, wherein the NaCl in the test sample is at a concentration of from about 10 mM to about 500 mM, or from about 50 mM to about 250 mM, or from about 100 mM to about 200 mM or about 102 mM.
Embodiment 139. The test sample of embodiment 137 or 138, wherein the KCl in the test sample is at a concentration of from about 0.5 mM to about 30 mM, or from about 1 mM to about 20 mM, or from about 2 mM to about 15 mM, or from about 4 mM to about 10 mM, or about 5 mM.
Embodiment 140. The test sample of any one of embodiments 137-139, wherein the MgCl₂ in the test sample is at a concentration of from about 0.5 mM to about 30 mM, or from about 1 mM to about 20 mM, or from about 2 mM to about 15 mM, or from about 4 mM to about 10 mM, or about 5 mM.
Embodiment 141. The test sample of any one of embodiments 131-140, wherein the buffering agent is selected from HEPES, IVIES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.
Embodiment 142. The test sample of any one of embodiments 131-141, wherein the buffering agent in the test sample is at a concentration of from about 4 mM to about 400 mM, or from about 10 mM to about 300 mM, or from about 20 mM to about 200 mM, or from about 30 mM to about 100 mM, or from 35 mM to about 50 mM or about 40 mM.
Embodiment 143. The test sample of any one of embodiments 131-142, wherein the chelating agent is selected from EDTA, EGTA, DTPA, BAPTA, DMPS and ALA.
Embodiment 144. The test sample of any one of embodiments 131-143, wherein the chelating agent in the test sample is at a concentration of about 0.1 mM to about 10 mM, or from about 0.5 mM to about 5 mM, or about 1 mM.
Embodiment 145. The test sample of any one of embodiments 131-144, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).
Embodiment 146. The test sample of any one of embodiments 131-145, wherein the nonionic surfactant is from about 0.01% to about 1% of the test sample, or about 0.02% to about 0.5% of the test sample, or from about 0.03% to about 0.1% of the test sample, or from about 0.04% to about 0.08% of the test sample, or about 0.05% of the test sample, volume by volume.
Embodiment 147. The test sample of any one of embodiments 131-146, wherein the total protein concentration of the test sample is determined with an bicinchoninic acid (BCA) assay, wherein the BCA assay is optionally a micro BCA assay.
Embodiment 148. The test sample of embodiment 147, wherein the assay comprises a bicinchoninic acid reagent or a bicinchonic acid reagent and, optionally, an alkaline tartrate-carbonate buffer and/or a copper sulfate solution.
Embodiment 149. The test sample of any one of embodiments 131-148, wherein the test sample comprises 40 mM HEPES, 102 mM NaCl, 5 mM KCl, 5 mM MgCl₂, 1 mM EDTA and 0.05% Tween 20.
Embodiment 150. The test sample of any one of embodiments 131-149, wherein the pH of the test sample is from about pH 5 to about pH 9, or from about pH 6 to about pH 8, or from about pH 7 to about pH 7.9, or about pH 7.5.
Embodiment 151. The test sample of any one of embodiments 131-150, wherein at least one capture reagent is an aptamer, and wherein the at least one aptamer comprises a 5-position modified pyrimidine.
Embodiment 152. The test sample of embodiment 151, wherein the 5-position modified pyrimidine comprises a hydrophobic moiety at the 5-position of the pyrimidine.
Embodiment 153. The test sample of embodiment 152, wherein the hydrophobic moiety is selected from a naphthyl moiety, a phenyl moiety, a tyrosyl moiety, an indole moiety and a morpholino moiety.
Embodiment 154. The test sample of embodiment 152 or 153, wherein the hydrophobic moiety is covalently linked to the 5-position of the based via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.
Embodiment 155. The test sample of any one of embodiments 131-154, wherein the biological sample is a urine sample.
Embodiment 156. The test sample of any one of embodiments 131-154, wherein the biological sample is a serum sample.
Embodiment 157. The test sample of any one of embodiments 131-156, wherein gel filtration chromatography was used to perform the buffer exchange on the biological sample in order to generate the buffer-exchanged biological sample.
Embodiment 158. A method for detecting a target protein in a test sample comprising:

contacting the test sample with at least one capture reagent, wherein the at least one capture reagent is capable of binding to the target protein to form a complex;

incubating the test sample with the at least one capture reagent under conditions that allow for the complex to form; and

determining the level of the target protein in the test sample by measuring the level of the at least one capture reagent, the complex, or the protein; wherein the level of the at least one capture reagent or the complex is a surrogate for the level of the target protein;

wherein, the test sample is generated by performing a buffer exchange of a biological sample with a formulation comprising a buffering agent, a salt, a chelating agent and a nonionic surfactant; and wherein, the total protein concentration of the test sample is about 70 μg/mL to about less than 100 μg/mL.

Embodiment 159. The method of embodiment 158, wherein the at least one protein capture reagent is an aptamer or an antibody.
Embodiment 160. The method of embodiment 158 or 159 comprising a plurality of capture reagents, where each capture reagent is an aptamer.
Embodiment 161. The method of any one of embodiments 158-160, wherein the total protein concentration of the test sample is about 70 μg/mL to 95 μg/mL; or about 70 μg/mL to 90 μg/mL; or about 70 μg/mL to 85 μg/mL; or about 70 μg/mL to 80 μg/mL; or about 70 μg/mL to 75 μg/mL or about 70 μg/mL.
Embodiment 162. The method of any one of embodiments 158-161, wherein the one or more salts are selected from a sodium salt, a potassium salt and a magnesium salt.
Embodiment 163. The method of any one of embodiments 158-161, wherein the one or more salts comprise a sodium salt, a potassium salt and a magnesium salt.
Embodiment 164. The method of embodiment 162 or 163, wherein the sodium salt is NaCl, the potassium salt is KCl and the magnesium salt is MgCl₂.
Embodiment 165. The method of embodiment 164, wherein the NaCl in the formulation is at a concentration of from about 10 mM to about 500 mM, or from about 50 mM to about 250 mM, or from about 100 mM to about 200 mM or about 102 mM.
Embodiment 166. The method of embodiment 164 or 165, wherein the KCl in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 167. The method of any one of embodiments 164-166, wherein the MgCl₂ in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 168. The method of any one of embodiments 158-167, wherein the buffering agent is selected from HEPES, MES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.
Embodiment 169. The method of any one of embodiments 158-168, wherein the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 50 mM or about 40 mM.
Embodiment 170. The method of any one of embodiments 158-169, wherein the chelating agent is selected from EDTA, EGTA, DTPA, BAPTA, DMPS and ALA.
Embodiment 171. The method of any one of embodiments 158-170, wherein the chelating agent in the formulation is at a concentration of about 0.1 mM to about 10 mM, or from about 0.5 mM to about 5 mM, or about 1 mM.
Embodiment 172. The method of any one of embodiments 158-171, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).
Embodiment 173. The method of any one of embodiments 158-172, wherein the nonionic surfactant is from about 0.01% to about 1% of the formulation, or about 0.02% to about 0.5% of the formulation, or from about 0.03% to about 0.1% of the formulation, or from about 0.04% to about 0.08% of the formulation, or about 0.05% of the formulation.
Embodiment 174. The method of any one of embodiments 158-173, wherein the total protein concentration of the test sample is measured with a bicinchoninic acid (BCA) assay, wherein the BCA assay is optionally a micro BCA assay.
Embodiment 175. The method of embodiment 174, wherein the assay comprises a bicinchoninic add reagent or a bicinchonic acid reagent and, optionally, an alkaline tartrate-carbonate buffer and/or a copper sulfate solution.
Embodiment 176. The method of any one of embodiments 158-175, wherein the formulation comprises 40 mM HEPES, 102 mM NaCl, 5 mM KCl, 5 mM MgCl₂, 1 mM EDTA and 0.05% Tween 20.
Embodiment 177. The method of any one of embodiments 158-175, wherein the formulation consists of 40 mM HEPES, 102 mM NaCl, 5 mM KCl, 5 mM MgCl₂, 1 mM EDTA and 0.05% Tween 20 at pH 7.5.
Embodiment 178. The method of any one of embodiments 158-177, wherein the pH of the formulation is from about pH 5 to about pH 9, or from about pH 6 to about pH 8, or from about pH 7 to about pH 7.9, or about pH 7.5.
Embodiment 179. The method of any one of embodiments 158-178, wherein the aptamer comprises a 5-position modified pyrimidine.
Embodiment 180. The method of embodiment 179, wherein the 5-position modified pyrimidine comprises a hydrophobic moiety at the 5-position of the pyrimidine.
Embodiment 181. The method of embodiment 180, wherein the hydrophobic moiety is selected from a naphthyl moiety, a phenyl moiety, a tyrosyl moiety, an indole moiety and a morpholino moiety.
Embodiment 182. The method of embodiment 180 or 181, wherein the hydrophobic moiety is covalently linked to the 5-position of the based via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.
Embodiment 183. The method of any one of embodiments 158-182, wherein the complex is a non-covalent complex.
Embodiment 184. The method of any one of embodiments 158-183, wherein the at least one capture reagent is attached to a first solid support before contacting the test sample or the capture reagent is attached to a first solid support after contacting the test sample.
Embodiment 185. The method of any one of embodiments 158-184, further comprising attaching the complex to a first solid support via the capture reagent.
Embodiment 186. The method of embodiment 185, further comprising releasing complex from the first solid support and attaching the complex to a second solid support.
Embodiment 187. The method of embodiment 186, wherein the complex is attached to the second solid support via the protein.
Embodiment 188. The method of any one of embodiments 158-187, further comprising adding a competitor molecule to the test sample and/or adding a competitor molecule and diluting the test sample.
Embodiment 189. The method of embodiment 188, wherein the competitor molecule is a polyanionic competitor.
Embodiment 190. The method of embodiment 189, wherein the polyanionic competitor is selected from an oligonucleotide, polydextran, DNA, heparin and dNTPs.
Embodiment 191. The method of embodiment 190, wherein the polyanionic competitor is a polydextran, and wherein the polydextran is dextran sulfate.
Embodiment 192. The method of embodiment 190, wherein the polyanionic competitor is an oligonucleotide, and wherein the oligonucleotide comprises one or more 5-position modified pyrimidines.
Embodiment 193. The method of any one of embodiments 158-192, wherein the biological sample is urine.
Embodiment 194. The method of any one of embodiments 158-192, wherein the biological sample is serum.
Embodiment 195. The method of any one of embodiments 100-130 or 158-194, wherein the buffer exchange is performed using gel filtration chromatography.
Embodiment 196. The method of any one of embodiments 100-130 or 158-195, further comprising measuring the protein concentration of the biological sample prior to the performing of the buffer exchange.
Embodiment 197. The method of embodiment 196, wherein the protein concentration of the biological sample is measured with an assay selected from the group consisting of a fluorescence readout assay, a bicinchoninic acid (BCA) assay, a micro BCA assay, a Lowry assay, and an ELISA assay.
Embodiment 198. The method of embodiment 197, wherein the assay is a BCA assay or a micro BCA assay.
Embodiment 199. The method of embodiment 198, wherein the assay comprises a bicinchoninic acid reagent or a bicinchonic acid reagent and, optionally, an alkaline tartrate-carbonate buffer and/or a copper sulfate solution.
Embodiment 200. The method of embodiment 197, wherein the assay is a fluorescence readout assay.
Embodiment 201. The method of embodiment 200, wherein the fluorescence readout assay comprises a reagent selected from a merocyanine dye and epicocconone.
Embodiment 202. A method for preparing a biological sample for detecting a protein comprising:

generating a test sample by performing a buffer exchange on the biological sample using a formulation comprising a buffering agent, one or more salts, a chelating agent and a nonionic surfactant; and

determining the total protein concentration of the biological sample or of the test sample; and

adjusting the total protein concentration of the biological sample or of the test sample, wherein the method does not comprise concentrating the total protein of the biological sample prior to performing the buffer exchange.

Embodiment 203. The method of embodiment 202, wherein the total protein concentration of the test sample is from about 2 μg/mL to about 60 μg/mL.
Embodiment 204. A method for preparing a biological sample for detecting a protein comprising:

determining the total protein concentration of the biological sample;

adjusting the total protein concentration of the biological sample to from about 2 μg/mL to about 60 μg/mL; and

generating a test sample by performing a buffer exchange on the biological sample using a formulation comprising a buffering agent, one or more salts, a chelating agent and a nonionic surfactant.

Embodiment 205. The method of any one of embodiments 202-204, comprising generating a plurality of test samples by performing a buffer exchange on a plurality of biological samples, wherein the total protein concentration of each test sample is about the same.
Embodiment 206. The method of any one of embodiments 202-205, comprising generating a plurality of test samples by performing a buffer exchange on a plurality of biological samples, wherein the total protein concentration of each test sample is the same.
Embodiment 207. A method of preparing a biological sample for detecting a protein comprising:

determining the protein concentration of the biological sample; and

if the total protein concentration of the biological sample is not in the range of from about 2 μg/mL to about 60 μg/mL, adjusting the total protein concentration of the biological sample to from about 2 μg/mL to about 60 μg/mL; and

generating a test sample by performing a buffer exchange on the biological sample using a formulation comprising a buffering agent, one or more salts, a chelating agent and a nonionic surfactant.

Embodiment 208. The method of embodiment 207, wherein if the total protein concentration of the biological sample is not in the range of from about 2 μg/mL to about 50 μg/mL; or from about 2 μg/mL to about 45 μg/mL; or from about 4 μg/mL to about 40 μg/mL; or from about 8 μg/mL to about 40 μg/mL; or from about 10 μg/mL to about 40 μg/mL; or from about 10 μg/mL to about 30 μg/mL; or less than 40 μg/mL; or less than 30 μg/mL; or less than 20 μg/mL; or about 15 μg/mL; or from about 10 μg/mL to about 20 μg/mL, the method comprises adjusting the total protein concentration of the biological sample to the range.
Embodiment 209. The method of any one of embodiments 202-208, wherein the one or more salts are each independently selected from a sodium salt, a potassium salt and a magnesium salt.
Embodiment 210. The method of any one of embodiments 202-208, wherein the one or more salts comprise a sodium salt, a potassium salt and a magnesium salt.
Embodiment 211. The method of any one of embodiments 209 or 210, wherein the sodium salt is NaCl, the potassium salt is KCl and the magnesium salt is MgCl₂.
Embodiment 212. The method of embodiment 211, wherein the NaCl in the formulation is at a concentration of from about 10 mM to about 500 mM, or from about 50 mM to about 250 mM, or from about 100 mM to about 200 mM, or about 102 mM.
Embodiment 213. The method of embodiment 211 or 212, wherein the KCl in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 214. The method of any one of embodiments 211-213, wherein the MgCl₂ in the formulation is at a concentration of from about 0.5 mM to about 30 mM, or from about 1 mM to about 20 mM, or from about 2 mM to about 15 mM, or from about 4 mM to about 10 mM, or about 5 mM.
Embodiment 215. The method of any one of embodiments 202-214, wherein the buffering agent is selected from HEPES, MES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.
Embodiment 216. The method of embodiment 215, wherein the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 50 mM, or about 40 mM.
Embodiment 217. The method of any one of embodiments 202-216, wherein the chelating agent is selected from EDTA, EGTA, DTPA, BAPTA, DMPS and ALA.
Embodiment 218. The method of embodiment 217, wherein the chelating agent in the formulation is at a concentration of about 0.1 mM to about 10 mM, or from about 0.5 mM to about 5 mM, or about 1 mM.
Embodiment 219. The method of any one of embodiments 202-218, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).
Embodiment 220. The method of embodiment 219, wherein the nonionic surfactant is from about 0.01% to about 1% of the formulation, or about 0.02% to about 0.5% of the formulation, or from about 0.03% to about 0.1% of the formulation, or from about 0.04% to about 0.08% of the formulation, or about 0.05%, or about 0.1% of the formulation, volume for volume.
Embodiment 221. The method of any one of embodiments 202-220, wherein the total protein concentration of the test sample is from about 2 μg/mL to about 50 μg/mL; or from about 2 μg/mL to about 45 μg/mL; or from about 4 μg/mL to about 40 μg/mL; or from about 8 μg/mL to about 40 μg/mL; or from about 10 μg/mL to about 40 μg/mL; or from about 10 μg/mL to about 30 μg/mL; or less than 40 μg/mL; or less than 30 μg/mL; or less than 20 μg/mL; or about 15 μg/mL; or from about 10 μg/mL to about 20 μg/mL.
Embodiment 222. The method of any one of embodiments 203-221, wherein the total protein concentration of the test sample or the biological sample is determined with an assay selected from the group consisting of a fluorescence readout assay, a Bradford assay, a bicinchoninic acid (BCA) assay, a Lowry assay and an ELISA assay.
Embodiment 223. The method of embodiment 222, wherein the fluorescence readout assay comprises a reagent selected from a merocyanine dye and epicocconone.
Embodiment 224. The method of any one of embodiments 202-223, wherein the formulation comprises 40 mM HEPES, 102 mM NaCl, 5 mM KCl, 5 mM MgCl₂, 1 mM EDTA and 0.05% Tween-20.
Embodiment 225. The method of embodiment any one of embodiments 202-224, wherein the pH of the formulation is from about pH 5 to about pH 9, or from about pH 6 to about pH 8, or from about pH 7 to about pH 7.9, or about pH 7.5.
Embodiment 226. The method of any one of embodiments 202-225, wherein the formulation consists of 40 mM HEPES, 102 mM NaCl, 5 mM KCl, 5 mM MgCl₂, 1 mM EDTA and 0.05% Tween-20 and has a pH of 7.5.
Embodiment 227. The method of any one of embodiments 202-226, wherein the buffer exchange is not performed with ultrafiltration.
Embodiment 228. The method of any one of embodiments 202-227, wherein the buffer exchange is performed with gel filtration chromatography.
Embodiment 229. The method of any one of embodiments 202-228, wherein the biological sample is a urine sample.
Embodiment 230. The method of any one of embodiments 202-228, wherein the biological sample is a serum sample.
Embodiment 231. The method of any one of embodiments 202-230, wherein the method does not comprise a concentrating the biological sample.
Embodiment 232. A method for preparing a biological sample for detecting a protein comprising:

generating a test sample by performing a buffer exchange on the biological sample using a formulation comprising a buffering agent, one or more salts, a chelating agent and a nonionic surfactant; and

determining the total protein concentration of the biological sample or of the test sample; and

adjusting the total protein concentration of the biological sample or of the test sample, wherein the method does not comprise concentrating the total protein of the biological sample prior to performing the buffer exchange,

optionally wherein (i) the total protein concentration is adjusted before performing the buffer exchange or (ii) the total protein concentration is adjusted after performing the buffer exchange.

The method of embodiment 232 may further comprise any applicable feature of the preceding embodiments, mutandis mutatis. Any of the foregoing embodiments may be performed in an order in which the total protein adjustment (and measurement if applicable) is performed before or after buffer exchange, or altered such that the total protein adjustment (and measurement if applicable) is performed before or after buffer exchange, as appropriate. Any method disclosed or claimed herein should be interpreted as encompassing both orders, i.e., in which the total protein adjustment (and measurement if applicable) is performed before or after buffer exchange, unless the language clearly requires otherwise.

### Certain Methods

In some embodiments, methods disclosed herein facilitate standardization of total protein concentrations in biological samples or test samples while limiting inter-sample variability in analyte measurements and expanding the linear ranges of the analytes in the samples. In some embodiments, the methods comprise performing buffer exchange. In some such embodiments, the buffer exchange allows both adjustment of the total protein concentration of the biological sample and control of salt concentrations of the biological sample. Variation in salt concentrations in a biological sample may cause significant variation in measurement of analytes within the sample. The ability to adjust the total protein concentration and salt concentrations limits such inter-sample variability. In some embodiments, the methods herein expand the linear range of analyte concentrations, allowing more reliable measurement of analytes compared to measurement of analytes in biological samples or test samples that are not buffer-exchanged.

In some embodiments, methods herein comprise determining total protein concentration of the biological sample. In some such embodiments, the total protein concentration of the biological sample is determined prior to buffer exchange of the biological sample. In some embodiments, methods herein comprise determining total protein concentration of the test sample. In some such embodiments, the total protein concentration of the test sample is determined after buffer exchange of the biological sample. In some embodiments, the total protein concentration is determined using a fluorescence readout assay. In some embodiments, the total protein concentration is determined using a BCA assay. In some embodiments, the total protein concentration of the test sample is determined to be a certain value or within a certain range and is not adjusted. In some embodiments, the total protein concentration is determined to not be a certain value or is outside a certain range and is adjusted. In some embodiments, the range is not the same for every type of assay used to determine the total protein concentration. In some embodiments, methods herein comprise generating a plurality of test samples from a plurality of biological samples, wherein the total protein concentration of some test samples or of some biological samples is adjusted and the total protein concentration of other test samples or of other biological samples is not adjusted.

In some embodiments, the methods herein do not comprise ultrafiltration or concentration of the total protein of a biological sample. In some such embodiments, buffer exchange is performed using gel filtration chromatography.

### Certain Test Samples

In some embodiments, buffer-exchanged test samples disclosed herein comprise a total protein concentration less than about 60 μg/mL, between 60 and 100 μg/mL, or between 70 and 100 μg/mL. Some such embodiments comprise HEPES, NaCl, KCl, MgCl₂, Tween, and EDTA.

### Inter-Sample Variability in Biological Samples

Variability in total protein concentration from biological samples, such as random untimed ‘spot’ urine specimens, represents a challenge for proteomic analyses. The normal range for total protein in a spot urine sample is from about 1 to 14 mg/dL with from about 50 to 80 mg excreted per day when at rest. Urinary protein concentration tends to increase with age, exercise, and standing posture. In nephrotic syndromes total excretion of protein may exceed 3.5 grams per day.

Variability in protein concentrations between urine specimens was reported by Thomas et al. (Thomas, C. E., Sexton W., Benson K., Sutphen R., and J. Koomen. (2010). Urine collection and processing for protein biomarker discovery and quantification. Cancer Epidemiol Biomarkers Prev 19(4):953-959). In this study, all voids were collected from one individual over 10 consecutive days. The total protein concentration was determined for each and was found to vary by over 100-fold across specimens. The coefficient of variation (CV) was lowest for 24-hour pools (39%) and for first morning voids (41%). The greatest CV was observed for the second void of the day (54%) and for all spot collections (61%).

Composition and concentrative properties of human urine. Prepared for the National Aeronautics and Space Administration by McDonnell Douglas Astronautics Company Huntington Beach, Calif.) showed that 68 small molecule constituents make up 99% of the dissolved solids in urine. These 68 molecules add up to approximately 36.8 grams per liter in a typical urine sample. The top 10 molecules, ranked from highest to lowest by mass per unit volume, are urea, chloride, sodium, potassium, creatinine, inorganic sulfur, hippuric acid, phosphorus, citric acid, and glucuronic acid. However, between urine specimens, the absolute concentration of any of these 68 components varies considerably. Table 1 below summarizes some of the observed differences among several urine samples.

TABLE 1
Variability in the Characteristics and Composition of Urine Samples

| Characteristic/Composition | Urine |
|---|---|
| pH | 4.5-8.0 |
| Sodium | 117-840 mg/dL |
| Chloride | 187-840 mg/dL |
| Magnesium | 2-20.5 mg/dL |
| Calcium | 3-39 mg/dL |
| Phosphorus | 47-107 mg/dL |
| Potassium | 75-261 mg/dL |

The variability in urine total protein concentrations from inter-subject comparisons and inter-sample comparison from the same subject, in addition to the variability in pH, salt concentration and other components make urine a challenging matrix for proteomic measurements in biomarker discovery and diagnostics because the measurement of protein levels may be impacted by the presence, specifically the concentration of these components (e.g., salts and other solids) in urine. Effectively, a sample with different levels of these components yet the same concentration of protein, assayed for protein levels, will give different protein measurements based on the levels of the different components of the sample.

The foregoing and other objects, features, and advantages of the invention will become more apparent from the following detailed description, which proceeds with reference to the accompanying figures.

### DESCRIPTION OF THE FIGURES

FIGS. 1A and 1B show the change in analyte measurements as a function of NaCl concentration. Both FIGS. 1A and 1B show the same data presented differently. FIG. 1A shows on the x-axis the additional NaCl (mM) concentration to the SB17 buffer formulations, which starts with 102 mM NaCl, and the y-axis is the Log 2-fold change in RFU (relative fluorescent units), which correlates with the amount of analyte present in the sample, relative to the control buffer condition (SB17 buffer). FIG. 1B shows on the x-axis the additional NaCl (mM) concentration to the SB17 buffer, which starts with 102 mM NaCl, and the y-axis is the percent of analytes that are increased or decreased as a function of NaCl concentration.

FIGS. 2A and 2B show the change in analyte measurements as a function of MgCl₂ concentration. Both FIGS. 2A and 2B are the same data presented differently. FIG. 2A shows on the x-axis the additional MgCl₂ (mM) concentration to the SB17 buffer formulations, which starts with 5 mM MgCl₂, and the y-axis is the Log 2-fold change in RFU (relative fluorescent units), which correlates with the amount of analyte present in the sample, relative to the control buffer condition (SB17 buffer). FIG. 2B shows on the x-axis the additional MgCl₂ (mM) concentration to the SB17 buffer, which starts with 5 mM MgCl₂, and the y-axis is the percent of analytes that are increased or decreased as a function of MgCl₂ concentration.

FIGS. 3A and 3B show the change in analyte measurements as a function of Urea concentration. Both FIGS. 3A and 3B are the same data presented differently. FIG. 3A shows on the x-axis the addition of Urea (mM) concentration to the SB17 buffer formulations, which starts with 0 mM, and the y-axis is the Log 2-fold change in RFU (relative fluorescent units), which correlates with the amount of analyte present in the sample, relative to the control buffer condition (SB17 buffer). FIG. 3B shows on the x-axis the additional Urea (mM) concentration to the SB17 buffer, which starts with 0 mM, and the y-axis is the percent of analytes that are increased or decreased as a function of Urea concentration.

FIG. 4 shows a visualization of the linear range for 2,298 analytes measured in this titration. The black bars span the lower and upper limits of the linear range for each analyte (x-axis) as a function of the total protein concentration in μg/mL (y-axis) of the sample.

FIG. 5 shows the percent of the 2,298 analytes (x-axis) that are in the linear range as a function of total protein concentration in μg/mL (y-axis) of the sample.

FIG. 6 shows the effect that sample buffer exchange has on analyte linearity in a proteomic assay. Two different analytes were measured in the assay with one sample being subject to a buffer exchange and the other not subject to a buffer exchange (control). A titration based on total protein concentration (x-axis) of the sample was performed and the level of analytes, as measured by RFU, was plotted on a Log 2 scale (y-axis). For the ALDO protein analyte in the control sample, a linear range is observed at lower total protein concentrations; however beyond about 30 μg/mL, the RFU measurements being to decrease. When the sample is subject to a buffer exchange, the linear range of the same analyte (ALDO) extends well into a total protein concentration for the sample of 130 μg/mL. Likewise, For the ANXA4 protein analyte in the control sample, a linear range is observed at lower total protein concentrations; however beyond about 16 μg/mL, the RFU measurements being to level off or plateau. When the sample is subject to a buffer exchange, the linear range of the same analyte (ANXA4) extends well into a total protein concentration for the sample of 130 μg/mL.

FIGS. 7A and 7B graph the linearity of the analytes measured in the assay as a function of total protein concentration of the sample. Both FIGS. 7A and 7B are the same data presented differently. The analytes are categorized as being in the linear range, being above the linear range or below the linear range. FIG. 7A graphs the number of analytes and their categorization (above, below or in the linear range) (y-axis) as a function of the total protein concentration of the sample. FIG. 7B graphs the percent of analytes and their categorization (above, below or in the linear range) (y-axis) as a function of the total protein concentration of the sample.

FIG. 8. Certain exemplary 5-position modified uridines and cytidines that may be incorporated into aptamers.

FIG. 9. Certain exemplary modifications that may be present at the 5-position of uridine. The chemical structure of the C-5 modification includes the exemplary amide linkage that links the modification to the 5-position of the uridine. The 5-position moieties shown include a phenyl moiety (e.g., Bn, PE and a PP), a naphthyl moiety (e.g., Nap, 2Nap, NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), a benzofuranyl moiety (e.g., BF), an indole moiety (e.g, Trp) and a hydroxypropyl moiety (e.g., Thr).

FIG. 10. Certain exemplary modifications that may be present at the 5-position of cytidine. The chemical structure of the C-5 modification includes the exemplary amide linkage that links the modification to the 5-position of the cytidine. The 5-position moieties shown include a phenyl moiety (e.g., Bn, PE and a PP), a naphthyl moiety (e.g., Nap, 2Nap, NE, and 2NE) and a tyrosyl moiety (e.g., a Tyr).

FIG. 11 shows a graph of the number of analytes in urine samples in the linear range as measured by an aptamer-based proteomic assay, as a function of total protein concentration as measured by a micro BCA assay.

FIGS. 12A and 12B show the number (FIG. 12A) or mean number (FIG. 12B) of analytes in urine samples in the linear range as measured by an aptamer-based proteomic assay, as a function of total protein concentration, from 1-150 μg/mL, as measured by a micro BCA assay.

FIG. 12A shows the results for ten individual urine samples. FIG. 12B shows combined results for the same ten individual urine samples.

### DETAILED DESCRIPTION

### I. Terms and Methods

While the invention will be described in conjunction with certain representative embodiments, it will be understood that the invention is defined by the claims, and is not limited to those embodiments.

One skilled in the art will recognize many methods and materials similar or equivalent to those described herein may be used in the practice of the present invention. The present invention is in no way limited to the methods and materials described.

Unless defined otherwise, technical and scientific terms used herein have the meaning commonly understood by one of ordinary skill in the art to which this invention belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods, devices, and materials similar or equivalent to those described herein can be used in the practice of the invention, certain methods, devices, and materials are described herein.

All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include the plural, unless the context clearly dictates otherwise, and may be used interchangeably with “at least one” and “one or more.” Thus, reference to “an aptamer” includes mixtures of aptamers, reference to “a probe” includes mixtures of probes, and the like.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements may include other elements not expressly listed.

It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as fractions thereof unless the context clearly dictates otherwise.

Any concentration range, percentage range, ratio range, or integer range is to be understood to include the endpoints and the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

The use of the alternative (e.g., “or”) should be understood to mean either one, both, or any combination thereof of the alternatives.

In order to facilitate review of the various embodiments of the disclosure, the following explanations of specific terms are provided:

About: The term “about” in the context of a numerical value or range means the recited numerical value or range plus or minus 10% of the numerical value or range, unless otherwise indicated.

Antibody: The term “antibody” refers to full-length antibodies of any species and fragments and derivatives of such antibodies that retain the ability to bind to antigen, including Fab fragments, F(ab′)2 fragments, single chain antibodies, Fv fragments, and single chain Fv fragments. The term “antibody” also includes synthetically-derived antibodies, such as phage display-derived antibodies and fragments, affybodies and nanobodies.

Aptamer: As used herein, an “aptamer” refers to a nucleic acid that has a specific binding affinity for a target molecule, wherein the binding of the aptamer to the target molecule does not comprise Watson-Crick base pairing. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. The plural “aptamers” refers to more than one such set of molecules. Different aptamers can have either the same or different numbers of nucleotides. Aptamers can be DNA or RNA or chemically modified nucleic acids, and can be single-stranded, double-stranded, or contain both single- and double-stranded regions, and can include higher ordered structures. An aptamer can also comprise a photoreactive or chemically reactive functional group to allow it to be covalently linked to its corresponding target. Any of the aptamer methods disclosed herein can include the use of two or more aptamers that specifically bind the same target molecule. As further described below, an aptamer may include a tag. If an aptamer includes a tag, all copies of the aptamer need not have the same tag. Moreover, if different aptamers each include a tag, each different aptamer can have either the same tag or a different tag.

BCA assay or bicinchoninic acid assay or bicinchonic assay: As used here, “BCA assay,” “bicinchoninic acid assay,” and “bicinchonic acid assay” are used interchangeably to refer to any assay, kit, reagent, or composition comprising bicinchoninic acid that can be used to determine protein concentration in a sample. BCA assays include, but are not limited to, micro BCA assays. An exemplary micro BCA assay is described in Example 6.

Biological Sample or Biological Matrix: As used herein, “biological sample” and “biological matrix” refer to any material, solution, or mixture obtained from an organism. This includes blood (including whole blood, leukocytes, peripheral blood mononuclear cells, plasma, and serum), sputum, breath, urine, semen, saliva, meningeal fluid, amniotic fluid, glandular fluid, lymph fluid, nipple aspirate, bronchial aspirate, synovial fluid, joint aspirate, cells, a cellular extract, and cerebrospinal fluid. This also includes experimentally separated fractions of all of the preceding. The terms “biological sample” and “biological matrix” also include materials, solutions, or mixtures containing homogenized solid material, such as from a stool sample, a tissue sample, or a tissue biopsy, for example. The terms “biological sample” and “biological matrix” also include materials, solutions, or mixtures derived from a cell line, tissue culture, cell culture, bacterial culture, viral culture or cell free biological system (e.g. IVTT).

Level: As used herein, “target protein level,” “analyte level” and “level” refer to a measurement that is made using any analytical method for detecting the analyte (such as a target protein) in a biological sample and that indicates the presence, absence, absolute amount or concentration, relative amount or concentration, titer, level, expression level, ratio of measured levels, or the like, of, for, or corresponding to the analyte in the biological sample. The exact nature of the “level” depends on the specific design and components of the particular analytical method employed to detect the analyte.

Buffering Agent: As used herein, “buffering agent” means a chemical or combination of chemicals that can adjust and/or maintain the pH of a solution in which they are dissolved. In some embodiments, a buffering agent is a weak acid. In some embodiments, a buffering agent is a weak base. In some embodiments, a buffering agent is a weak acid and a weak base. In some embodiments, buffering agents are salts. In some such embodiments, a buffering agent is a salt of a weak acid and of a weak base. In some embodiments, a buffering agent is zwitterionic. In some embodiments, a buffering agent is selected from 4-(2-hydroxyethyl)-1-piperazineethanesulfonic acid (HEPES), 2-(N-morpholino)ethanesulfonic acid (MES), Bis-tris methane, N-(2-acetamido)iminodiacetic acid (ADA), N-(carbamoylmethyl)-2-aminoethane sulfonic acid (ACES), Bis-tris propane, piperazine-N,N′-bis(2-ethanesulfonic acid) (PIPES), 3-morpolino-2-hydroxypropanesulfonic acid (MOPSO), Cholamine chloride, 3-(N-morpholino)propanesulfonic acid (MOPS), N,N-bis(2-hydroxyethyl)-2-aminoethanesulfonic acid (BES), N-[tris(hydroxymethyl)methyl]-2-aminoethanesulfonic acid (TES), 3-(N,N-Bis[2-hydroxyethyl]amino)-2-hydroxypropanesulfonic acid (DIPSO), 4-(N-Morpholino)butanesulfonic acid (MOB), Acetamidoglycine, 2-Hydroxy-3-[tris(hydroxymethyl)methylamino]-1-propanesulfonic acid (TAPSO), N,N-Diethylethanamine (TEA), Piperazine-N,N′-bis(2-hydroxypropanesulfonic acid) (POPSO), 4-(2-hydroxyethyl)piperazine-1-(2-hydroxypropane-3-sulfonic acid) hydrate (HEPPSO), 4-(2-Hydroxyethyl)-1-piperazinepropanesulfonic acid (EPPS), Tricine, Tris, Glycinamide, Glycylglycine, N-(2-Hydroxyethyl)piperazine-N′-(4-butanesulfonic acid) (HEPBS), Bicine, N-[Tris(hydroxymethyl)methyl]-3-aminopropanesulfonic acid (TAPS), 2-(Cyclohexylamino)ethanesulfonic acid; 2-(N-Cyclohexylamino)ethanesulfonic acid (CHES), β-Aminoisobutyl alcohol hydrochloride (AMP), N-(1,1-Dimethyl-2-hydroxyethyl)-3-amino-2-hydroxypropanesulfonic acid (AMPSO), N-cyclohexyl-2-hydroxyl-3-aminopropanesulfonic acid) (CAPSO), 3-(Cyclohexylamino)-1-propanesulfonic acid (CAPS) and 4-(Cyclohexylamino)-1-butanesulfonic acid (CABS).

Buffer-exchanged Sample: As used herein, a “buffer-exchanged sample” upon which performing a buffer exchange step has been performed. In some embodiments, a buffer exchange step comprises changing the identities and/or concentrations of one or more buffer components. Such buffer components include but are not limited to buffering agents, salts, chelating agents, and surfactants. In some embodiments, the total protein concentration of a buffer-exchanged sample is the same as the total protein concentration of the sample prior to the performance of the buffer exchange step. In some embodiments, a buffer-exchanged sample is a buffer-exchanged biological sample.

C-5 Modified Pyrimidine: As used herein, the term “C-5 modified pyrimidine” refers to a pyrimidine with a modification at the C-5 position. Examples of a C-5 modified pyrimidine include those described in U.S. Pat. Nos. 5,719,273; 5,945,527; 9,163,056; and Dellafiore et al., 2016, Front. Chem., 4:18. Examples of a C-5 modification include substitution of deoxyuridine at the C-5 position with a substituent independently selected from: benzylcarboxyamide (alternatively benzylaminocarbonyl) (Bn), naphthylmethylcarboxyamide (alternatively naphthylmethylaminocarbonyl) (Nap), tryptaminocarboxyamide (alternatively tryptaminocarbonyl) (Trp), phenethylcarboxyamide (alternatively phenethylamino carbonyl) (Pe), thiophenylmethylcarboxyamide (alternatively thiophenylmethylaminocarbonyl) (Th) and isobutylcarboxyamide (alternatively isobutylaminocarbonyl) (iBu) as illustrated immediately below.

Chemical modifications of a C-5 modified pyrimidine can also be combined, singly or in any combination, with 2′-position sugar modifications, modifications at exocyclic amines, and substitution of 4-thiouridine and the like.

Representative C-5 modified pyrimidines include: 5-(N-benzylcarboxyamide)-2′-deoxyuridine (BndU), 5-(N-benzylcarboxyamide)-2′-O-methyluridine, 5-(N-benzylcarboxyamide)-2′-fluorouridine, 5-(N-isobutylcarboxyamide)-2′-deoxyuridine (iBudU), 5-(N-isobutylcarboxyamide)-2′-O-methyluridine, 5-(N-phenethylcarboxyamide)-2′-deoxyuridine (PedU), 5-(N-thiophenylmethylcarboxyamide)-2′-deoxyuridine (ThdU), 5-(N-isobutylcarboxyamide)-2′-fluorouridine, 5-(N-tryptaminocarboxyamide)-2′-deoxyuridine (TrpdU), 5-(N-tryptaminocarboxyamide)-2′-O-methyluridine, 5-(N-tryptaminocarboxyamide)-2′-fluorouridine, 5-(N-[1-(3-trimethylamonium)propyl]carboxyamide)-2′-deoxyuridine chloride, 5-(N-naphthylmethylcarboxyamide)-2′-deoxyuridine (NapdU), 5-(N-naphthylmethylcarboxyamide)-2′-O-methyluridine, 5-(N-naphthylmethylcarboxyamide)-2′-fluorouridine or 5-(N-[1-(2,3-dihydroxypropyl)]carboxyamide)-2′-deoxyuridine).

Nucleotides can be modified either before or after synthesis of an oligonucleotide. A sequence of nucleotides in an oligonucleotide may be interrupted by one or more non-nucleotide components. A modified oligonucleotide may be further modified after polymerization, such as, for example, by conjugation with any suitable labeling component.

As used herein, the term “at least one pyrimidine,” when referring to modifications of a nucleic acid, refers to one, several, or all pyrimidines in the nucleic acid, indicating that any or all occurrences of any or all of C, T, or U in a nucleic acid may be modified or not.

Capture Reagent: As used herein, a “capture agent” or “capture reagent” refers to a molecule that is capable of binding specifically to an analyte, such as a biomarker, protein and/or peptide. A “target protein capture reagent” refers to a molecule that is capable of binding specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents. In some embodiments, a capture reagent is selected from an aptamer and an antibody.

Chelating Agent: As used herein, a “chelating agent” means a chemical that can form a plurality of bonds to a single metal ion. In some embodiments, a chelating agent is selected from Ethylenediaminetetraacetic Acid (EDTA), Ethylene Glycol Tetraacetic Acid (EGTA), Diethylenetriaminepentaacetic acid (DTPA), 1,2-Bis(2-aminophenoxy)ethane-N,N,N′,N′-tetraacetic acid (BAPTA), 2,3-Dimercaptopropanesulfonic acid (DMPS).

Control Level: A “control level” of a target molecule refers to the level of the target molecule in the same sample type from an individual that does not have the disease or condition, or from an individual that is not suspected or at risk of having the disease or condition, or from an individual that has a non-progressive form of the disease or condition. Further, a “control level” may refer to a reference based on the average or what is considered within normal or healthy parameters. A “control level” may also refer to a reference level taken at a previous time and that is used to compare to a later measured or detected level of a target. For example, the level of a target may be detected at time point A, and then detected at time point B, where time point B is after time point A. In a more specific example, time point A may be considered time zero (0) or day zero (0) and time point B may be minutes (e.g., 10, 20, 30, 40, 50, 60 minutes after time point A), hours (e.g, 1, 2, 3, 4, 5, 6, 7, 8, 9, 10, 11, 12, 13, 14, 15, 16, 17, 18, 19, 20, 21, 22, 23 or 24 hours after time point A), days (e.g, 1, 2, 3, 4, 5, 6 or 7 days after time point A), weeks (e.g., 1, 2, 3 or 4 weeks after time point A), months (e.g., 1, 2, 3, 4, 5, 6, 7, 8, 9, 10, 11 or 12 months after time point A) and even years (e.g., 1, 2, 3, 4, 5, 6, 7, 8, 9, 10, 11, 12, 13, 14, 15, 20, 25, 30, 35, 40, 45, 50, 55 or 60 years after time point A) after time point A. A “control level” of a target molecule need not be determined each time the present methods are carried out, and may be a previously determined level that is used as a reference or threshold to determine whether the level in a particular sample is higher or lower than a normal level.

Correspondence correlation: “Correspondence correlation” or “concordance correlation coefficient” measures the agreement between two continuous variables X and Y (e.g., predicted, estimated or determined, and actual). The “correspondence correlation” evaluates the degree to which pairs fall on the 45° line, and contains measurements of accuracy and precision (or the “Lin's Condordance”). Additional information may be found in Lin, Biometrics, Vol. 45, No. 1 (March, 1989), 255-268, which is hereby incorporated by reference. Other methods for determining correlation that may be used herein includes, but are not limited to, Pearson correlation coefficient, the paired t-test, least squares analysis of slope (=1) and intercept (=0), the coefficient of variation and the intraclass correlation coefficient. In certain embodiments, the correspondence correlation is determined by the method selected from Lin's Concordance, Pearson correlation coefficient, the paired t-test, least squares analysis of slope (=1) and intercept (=0), the coefficient of variation and the intraclass correlation coefficient.

Detecting: As used herein, “detecting” or “determining” includes the use of both the instrument used to observe and record a signal corresponding to an analyte level and the material/s required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

Diagnose: “Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual.

Dilution: “Dilution”, “dilution series” and variations thereof encompass several different types of dilutions, including, but not limited to, step dilutions, serial dilutions and combinations thereof. By way of example for a step dilution, if the dilution factor is 1000 (1:1000 dilution), the user may first perform a 1:10 dilution (dilution factor of 10) followed by a 1:100 dilution (dilution factor of 100) using 1 part solute from the 1:10 dilution and 99 parts of diluent, thus resulting in a dilution factor of 1000 or 1:1000 dilution of the solute. A serial dilution includes a succession of step dilutions, each having the same dilution factor, where the diluted material from the previous step is used to make the subsequent dilution. By way of example for a serial dilution, to make a 5-point 1:2 serial dilution, entails using 1 part solute and combining with 1 part diluent to make the first dilution (1′ point of the 5-point) in the dilution series, followed by 1 part solute from the first dilution and combining with 1 part diluent to make the second dilution (2^{nd} point of the 5-point) of the serial dilution series, so on and so forth until you reach the fifth successive serial dilution.

Dilution Factor: “Dilution factor” refers to the ratio of the parts of solute to parts of diluent. For example, a dilution factor of 2 means a 1:2 dilution where there are 1 part solute and 1 part diluent for a total of 2 parts; and a dilution factor of 10 means a 1:10 dilution where there are 1 part solute and 9 parts diluent for a total of 10 parts.

Evaluate: “Evaluate”, “evaluating”, “evaluation”, and variations thereof encompass both “diagnose” and “prognose” and encompass determinations or predictions about the future course of a disease or condition in an individual has the disease or condition, as well as determinations and predictions about eh likelihood that a disease or condition will occur in an individual who has not previously been diagnosed with the disease or condition, as well as determinations or predictions regarding the likelihood that a disease or condition will recur in an individual who is in remission or is believed to have been cured of the disease. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered, or is being administered, to the individual.

Individual: As used herein, “individual” and “subject” are used interchangeably to refer to a test subject or patient. The individual can be a mammal or a non-mammal. In various embodiments, the individual is a mammal. A mammalian individual can be a human or non-human. In various embodiments, the individual is a human. A healthy or normal individual is an individual in which the disease or condition of interest is not detectable by conventional diagnostic methods.

Linear Regression: The term “linear regression”, as used herein, refers to an approach for modeling the relationship between a scalar dependent variable y and one or more explanatory variables denoted x. The case of one explanatory variable is called simple linear regression. For more than one explanatory variable, it is called multiple linear regression. In general, linear regression may be used to fit a predictive model to an observed data set of y and x values. After developing such a model, if additional value of x is then given without its accompanying value of y, the fitted model can be used to make a prediction of the value of y.

Marker: As used herein, “marker” and “biomarker” are used interchangeably to refer to a target molecule (or analyte) that indicates or is a sign of a normal or abnormal process in an individual or of a disease or other condition in an individual. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. In some embodiments, a biomarker is a target protein.

Modified: As used herein, the terms “modify”, “modified”, “modification”, and any variations thereof, when used in reference to an oligonucleotide, means that at least one of the four constituent nucleotide bases (i.e., A, G, T/U, and C) of the oligonucleotide is an analog or ester of a naturally occurring nucleotide. In some embodiments, the modified nucleotide confers nuclease resistance to the oligonucleotide. In some embodiments, the modified nucleotides lead to predominantly hydrophobic interactions of aptamers with protein targets resulting in high binding efficiency and stable co-crystal complexes. A pyrimidine with a substitution at the C-5 position is an example of a modified nucleotide. Modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers, in some embodiments, ranging from about 10 to about 80 kDa, PEG polymers, in some embodiments, ranging from about 20 to about 60 kDa, or other hydrophilic or hydrophobic biological or synthetic polymers. In one embodiment, modifications are of the C-5 position of pyrimidines. These modifications can be produced through an amide linkage directly at the C-5 position or by other types of linkages. Polynucleotides can also contain analogous forms of ribose or deoxyribose sugars that are generally known in the art, including 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

Nucleic acid: As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers.

Ordinary least squares: “Ordinary least squares” or “OLS” or “linear least squares”, as used herein, refers to a method for estimating the unknown parameters in a linear regression model. This method minimizes the sum of squared vertical distances between the observed responses in the dataset and the responses predicted by the linear approximation. The resulting estimator can be expressed by a simple formula, especially in the case of a single regressor on the right-hand side.

Prognose: “Prognose”, “prognosing”, “prognosis”, and variations thereof refer to the prediction of a future course of a disease or condition in an individual who has the disease or condition (e.g., predicting patient survival), and such terms encompass the evaluation of disease response during and/or after the administration of a treatment or therapy to the individual.

SELEX: The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific analyte, such as a target protein.

Sequence Identity: Sequence identity, as used herein, in the context of two or more nucleic acid sequences is a function of the number of identical nucleotide positions shared by the sequences (i.e., % identity=number of identical positions/total number of positions in the reference sequencer 100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST). For sequence comparisons, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith and Waterman, Adv. Appl. Math., 2:482, 1981, by the homology alignment algorithm of Needleman and Wunsch, J. Mol. Biol., 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel, F. M. et al., Current Protocols in Molecular Biology, pub. by Greene Publishing Assoc. and Wiley-Interscience (1987)). As used herein, when describing the percent identity of a nucleic acid, such as an aptamer, the sequence of which is at least, for example, about 95% identical to a reference nucleotide sequence, it is intended that the nucleic acid sequence is identical to the reference sequence except that the nucleic acid sequence may include up to five point mutations per each 100 nucleotides of the reference nucleic acid sequence. In other words, to obtain a desired nucleic acid sequence, the sequence of which is at least about 95% identical to a reference nucleic acid sequence, up to 5% of the nucleotides in the reference sequence may be deleted or substituted with another nucleotide, or some number of nucleotides up to 5% of the total number of nucleotides in the reference sequence may be inserted into the reference sequence (referred to herein as an insertion). These mutations of the reference sequence to generate the desired sequence may occur at the 5′ or 3′ terminal positions of the reference nucleotide sequence or anywhere between those terminal positions, interspersed either individually among nucleotides in the reference sequence or in one or more contiguous groups within the reference sequence.

SOMAmer: The term SOMAmer or SOMAmer reagent, as used herein, refers to an aptamer having improved off-rate characteristics. SOMAmer reagents are alternatively referred to as Slow Off-Rate Modified Aptamers, and may be selected via the improved SELEX methods described in U.S. Publication No. 20090004667, entitled “Method for Generating Aptamers with Improved Off-Rates”, which is incorporated by reference in its entirety. In some embodiments, a slow off-rate aptamer (including an aptamers comprising at least one nucleotide with a hydrophobic modification) has an off-rate (t½) of ≥2 minutes, ≥4 minutes, ≥5 minutes, ≥8 minutes, ≥10 minutes, ≥15 minutes, ≥30 minutes, ≥60 minutes, ≥90 minutes, ≥120 minutes, ≥150 minutes, ≥180 minutes, ≥210 minutes, or ≥240 minutes.

Substantially Equivalent: The phrase “substantially equivalent”, as used herein, denotes a sufficiently high degree of similarity between two numeric values such that one of skill in the art would consider the difference between the two values to be of little or no biological and/or statistical significance within the context of the characteristic measured by the values. More specifically, the difference between the two values (e.g., the difference between the reference value and the regression model reference value) is preferably less than about 25%, or less than about 20%, or less than about 15% or less than about 10% or less than about 5% or less than about 4% or less than about 3% or less than about 2.5% or less than about 2% or less than about 1%.

Target Molecule: “Target”, “target molecule”, and “analyte” are used interchangeably herein to refer to any molecule of interest that may be present in a sample. The term includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” refers to a set of copies of one type or species of molecule or multi-molecular structure. “Target molecules”, “targets”, and “analytes” refer to more than one type or species of molecule or multi-molecular structure. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

Test Sample: As used herein, “test sample” means a material, solution, or mixture that comprises or is derived from a biological sample. In some embodiments, a test sample is generated from a biological sample. In some embodiments, a test sample is generated from a biological sample or a solution comprising a biological sample by performing a buffer exchange on the biological sample or solution comprising the biological sample. As used herein, an “adjusted test sample” is a test sample to which an adjustment has been made such as a change in total protein concentration.

Total Protein: As used herein, “total protein” in a solution, buffer, or sample means all of the proteins in the solution, buffer, or sample. The total protein concentration, level, or amount takes into account all of the different types of proteins in the solution, buffer, or sample. In contrast, “target protein” or “analyte protein” refers to one specific protein in a solution, buffer, or sample.

The foregoing and other objects, features, and advantages of the invention will become more apparent from the following detailed description, which proceeds with reference to the accompanying figures.

Although methods and materials similar or equivalent to those described herein can be used in the practice or testing of the present disclosure, suitable methods and materials are described below. In addition, the materials, methods, and examples are illustrative only and not intended to be limiting.

### Detection and Determination of Analytes and Analyte Levels

An analyte level for the analytes described herein can be detected using any of a variety of known analytical methods. In one embodiment, an analyte level is detected using a capture reagent. In various embodiments, the capture reagent can be exposed to the analyte in solution or can be exposed to the analyte while the capture reagent is immobilized on a solid support. In some embodiments, the capture reagent contains a feature that is reactive with a secondary feature on a solid support. In these embodiments, the capture reagent can be exposed to the analyte in solution, and then the feature on the capture reagent can be used in conjunction with the secondary feature on the solid support to immobilize the analyte on the solid support. The capture reagent is selected based on the type of analysis to be conducted. Capture reagents include, but are not limited to, aptamers, antibodies, adnectins, ankyrins, other antibody mimetics and other protein scaffolds, chimeras, small molecules, F(ab′)₂ fragments, single chain antibody fragments, Fv fragments, single chain Fv fragments, nucleic acids, lectins, ligand-binding receptors, affybodies, nanobodies, imprinted polymers, avimers, peptidomimetics, hormone receptors, cytokine receptors, and synthetic receptors, including modifications and fragments of any of these.

In some embodiments, an analyte level is detected using an analyte/capture reagent complex.

In some embodiments, the analyte level is derived from the analyte/capture reagent complex and is detected indirectly, such as, for example, as a result of a reaction that is subsequent to the analyte/capture reagent interaction, but is dependent on the formation of the analyte/capture reagent complex.

In some embodiments, analytes are detected using a multiplexed format that allows for the simultaneous detection of two or more analytes in a biological sample. In some embodiments of the multiplexed format, capture reagents are immobilized, directly or indirectly, covalently or non-covalently, in discrete locations on a solid support. In some embodiments, a multiplexed format uses discrete solid supports where each solid support has a unique capture reagent associated with that solid support, such as, for example, quantum dots. In some embodiments, an individual device is used for the detection of each one of multiple analytes to be detected in a biological sample. Individual devices can be configured to permit each analyte in the biological sample to be processed simultaneously. For example, a microtiter plate can be used such that each well in the plate is used to analyze one or more of multiple analytes to be detected in a biological sample.

In one or more of the embodiments described herein, a fluorescent tag can be used to label a component of the analyte/capture reagent complex to enable the detection of the analyte level. In various embodiments, the fluorescent label can be conjugated to a capture reagent specific to any of the analytes described herein using known techniques, and the fluorescent label can then be used to detect the corresponding analyte level. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In some embodiments, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexFluor molecule, such as, for example, AlexaFluor 488, AlexaFluor 532, AlexaFluor 647, AlexaFluor 680, or AlexaFluor 700. In some embodiments, the dye molecule includes a first type and a second type of dye molecule, such as, e.g., two different AlexaFluor molecules. In some embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more embodiments, a chemiluminescence tag can optionally be used to label a component of the analyte/capture complex to enable the detection of an analyte level. Suitable chemiluminescent materials include any of oxalyl chloride, Rodamin 6G, Ru(bipy)₃²⁺, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In some embodiments, the detection method includes an enzyme/substrate combination that generates a detectable signal that corresponds to the analyte level. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In some embodiments, the detection method can be a combination of fluorescence, chemiluminescence, radionuclide and/or enzyme/substrate combinations that generate a measurable signal. In some embodiments, multimodal signaling could have unique and advantageous characteristics in analyte assay formats.

In some embodiments, the analyte levels for the analytes described herein can be detected using any analytical methods including, singleplex aptamer assays, multiplexed aptamer assays, singleplex or multiplexed immunoassays, mRNA expression profiling, miRNA expression profiling, mass spectrometric analysis, histological/cytological methods, etc. and as discussed below.

### Determination of Analyte Levels Using Aptamer-Based Assays

An analyte level for the analytes described herein can be detected using any of a variety of aptamer-based assays. Assays directed to the detection and quantification of physiologically significant molecules in biological samples and other samples are important tools in scientific research and in the health care field. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. Nos. 6,242,246, 6,458,543, and 6,503,715, each of which is entitled “Nucleic Acid Ligand Diagnostic Biochip”. Once the microarray is contacted with a sample, the aptamers bind to their respective target molecules present in the sample and thereby enable a determination of an analyte level corresponding to an analyte.

In one aspect, the aptamer may include up to about 100 nucleotides, up to about 95 nucleotides, up to about 90 nucleotides, up to about 85 nucleotides, up to about 80 nucleotides, up to about 75 nucleotides, up to about 70 nucleotides, up to about 65 nucleotides, up to about 60 nucleotides, up to about 55 nucleotides, up to about 50 nucleotides, up to about 45 nucleotides, up to about 40 nucleotides, up to about 35 nucleotides, up to about 30 nucleotides, up to about 25 nucleotides, and up to about 20 nucleotides. In a related aspect, the aptamer may be from about 25 to about 100 nucleotides in length (or from about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides in length) or from about 25 to 50 nucleotides in length (or from about 25, 26, 27, 28, 29, 30, 31, 32, 33, 34, 35, 36, 37, 38, 39, 40, 41, 42, 43, 44, 45, 46, 47, 48, 49 or 50 nucleotides in length).

An aptamer can be identified using any known method, including the SELEX process. Once identified, an aptamer can be prepared or synthesized in accordance with any known method, including chemical synthetic methods and enzymatic synthetic methods. In some embodiments, an aptamer comprises at least one nucleotide with a hydrophobic modification, such as a hydrophobic base modification, allowing for hydrophobic contacts with a target protein. Such hydrophobic contacts, in some embodiments, contribute to greater affinity and/or slower off-rate binding by the aptamer. In some embodiments, an aptamer comprises at least two, at least three, at least four, at least five, at least six, at least seven, at least eight, at least nine, or at least 10 nucleotides with hydrophobic modifications, where each hydrophobic modification may be the same or different from the others. In some embodiments, the hydrophobic base modification is a C-5 modified pyrimidine. Nonlimiting exemplary C-5 modified pyrimidines are described herein and/or are known in the art.

In some assay formats, the aptamers are immobilized on the solid support prior to being contacted with the sample. Under certain circumstances, however, immobilization of the aptamers prior to contact with the sample may not provide an optimal assay. For example, in some instances, pre-immobilization of the aptamers may result in inefficient mixing of the aptamers with the target molecules on the surface of the solid support, perhaps leading to lengthy reaction times and, therefore, extended incubation periods to permit efficient binding of the aptamers to their target molecules. Further, when photoaptamers are employed in the assay and depending upon the material utilized as a solid support, the solid support may tend to scatter or absorb the light used to affect the formation of covalent bonds between the photoaptamers and their target molecules. Moreover, depending upon the method employed, detection of target molecules bound to their aptamers can be subject to imprecision, since the surface of the solid support may also be exposed to and affected by any labeling agents that are used. Finally, immobilization of the aptamers on the solid support generally involves an aptamer-preparation step (i.e., the immobilization) prior to exposure of the aptamers to the sample, and this preparation step may affect the activity or functionality of the aptamers.

Aptamer assays or “aptamer-based assay(s)” that permit an aptamer to capture its target in solution and then employ separation steps that are designed to remove specific components of the aptamer-target mixture prior to detection have also been described (see, e.g., U.S. Publication No. 2009/0042206, entitled “Multiplexed Analyses of Test Samples”). The described aptamer assays enable the detection and quantification of a non-nucleic acid target (e.g., a protein target) in a test sample by detecting and quantifying a nucleic acid (i.e., an aptamer). The described methods create a nucleic acid surrogate (i.e., the aptamer) for detecting and quantifying a non-nucleic acid target, thus allowing the wide variety of nucleic acid technologies, including amplification, to be applied to a broader range of desired targets, including protein targets.

Aptamers can be constructed to facilitate the separation of the assay components from an aptamer analyte complex (or photoaptamer analyte covalent complex) and permit isolation of the aptamer for detection and/or quantification. In one embodiment, these constructs can include a cleavable or releasable element within the aptamer sequence. In other embodiments, additional functionality can be introduced into the aptamer, for example, a labeled or detectable component, a spacer component, or a specific binding tag or immobilization element. For example, the aptamer can include a tag connected to the aptamer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, which in some embodiments are carried out with all assay components in solution, may not require separation of sample and reagents prior to the detection of signal. These methods are rapid and easy to use.

In some embodiments, a method for signal generation takes advantage of anisotropy signal change due to the interaction of a fluorophore-labeled capture reagent with its specific analyte target. When the labeled capture reacts with its target, the increased molecular weight causes the rotational motion of the fluorophore attached to the complex to become much slower changing the anisotropy value. By monitoring the anisotropy change, binding events may be used to quantitatively measure the analytes in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a analyte level in a biological sample includes the following: (a) preparing a mixture by contacting the biological sample with an aptamer that includes a first tag and has a specific affinity for the analyte, wherein an aptamer affinity complex is formed when the analyte is present in the sample; (b) exposing the mixture to a first solid support including a first capture element, and allowing the first tag to associate with the first capture element; (c) removing any components of the mixture not associated with the first solid support; (d) attaching a second tag to the analyte component of the aptamer affinity complex; (e) releasing the aptamer affinity complex from the first solid support; (f) exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the analyte by detecting the aptamer component of the aptamer affinity complex. For example, protein concentration or levels in a sample may be expressed as relative fluorescence units (RFU), which may be a product of detecting the aptamer component of the aptamer affinity complex (e.g., aptamer complexed to target protein create the aptamer affinity complex). That is, for an aptamer-based assay, the protein concentration or level correlates with the RFU.

A nonlimiting exemplary method of detecting analytes in a biological sample using aptamers is described in Kraemer et al., PLoS One 6(10): e26332.

### Determination of Analyte Levels Using Immunoassays

In some embodiments, target protein or analyte protein levels are determined using an immunoassay. Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies and fragments thereof are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or level corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting analytes include analyte immunoprecipitation followed by quantitative methods that allow size and peptide level discrimination, such as gel electrophoresis, capillary electrophoresis, planar electrochromatography, and the like.

Methods of detecting and/or for quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 386 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Analyte Levels Using Gene Expression Profiling

In some embodiments, target mRNA levels are determined using a gene expression profiling method described herein. Measuring mRNA in a biological sample may, in some embodiments, be used as a surrogate for detection of the level of the corresponding protein in the biological sample. Thus, in some embodiments, an analyte or analyte panel described herein can be detected by detecting the appropriate RNA.

In some embodiments, mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

### Determination of Analyte Levels Using Mass Spectrometry Methods

In some embodiments, target protein or analyte protein levels are determined using mass spectometry. A variety of configurations of mass spectrometers can be used to detect analyte levels. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647R-716R (1998); Kinter and Sherman, New York (2000)).

Protein analytes and analyte levels can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)^{N}, atmospheric pressure photoionization mass spectrometry (APPI-MS), APPI-MS/MS, and APPI-(MS)^{N}, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein analytes and determination of analyte levels. Labeling methods include but are not limited to isobaric tag for relative and absolute quantitation (iTRAQ) and stable isotope labeling with amino acids in cell culture (SILAC). Capture reagents used to selectively enrich samples for candidate analyte proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Certain Detection Methods

### Aptamer Assay Method

An “Aptamer Assay Method” as used herein comprises or consists of the steps described below. In some embodiments, an aptamer-based assay is an Aptamer Assay Method. Unless indicated otherwise, all steps were performed at room temperature.

### Preparation of Aptamer Master Mix Solutions.

Aptamers were grouped into three unique mixes, Dil1, Dil2 and Dil3 and corresponding to the sample dilutions of 20%, 0.5% and 0.005%, respectively. The assignment of an aptamer to a mix was empirically determined by assaying a dilution series of matching plasma and serum samples with each aptamer and identifying the sample dilution that gave the largest linear range of signal. The segregation of aptamers and mixing with different dilutions of the sample (20%, 0.5% or 0.005%) allow the assay to span a 10⁷-fold range of protein concentrations. The stock solutions for aptamer master mix were prepared in HE-Tween buffer (10 mM HEPES, pH 7.5, 1 mM EDTA, 0.05% Tween 20) at 4 nM each aptamer and stored frozen at −20° C. Approximately, 4271 aptamers were mixed in Dil1 mix, ˜828 aptamers in Dil2 and ˜173 aptamers in Dil3 mix. Before use, stock solutions were diluted in HE-Tween buffer to a working concentration of 0.55 nM each aptamer and aliquoted into individual use aliquots. Before using aptamer master mixes for Catch-0 plate preparation, working solutions were heat-cooled to refold aptamers by incubating at 95° C. for 10 minutes and then at 25° C. for at least 30 minutes before use.

### Catch-0 Plate Preparation.

60 μL of Streptavidin Mag Sepharose 10% slurry (GE Healthcare, 28-9857) were dispensed into each well of the 96-well plates (Thermo Scientific, AB-0769). Beads were washed once with 175 μL of the Assay Buffer, which comprises 40 mM HEPES, 100 or 102 mM NaCl, 5 mM KCl, 5 mM MgCl₂, 1 mM EDTA, and 0.05% or 0.1% (v/v) Tween-20, at pH 7.5, and then 100 μL of the heat-cooled aptamer master mix was added to each well. Plates were incubated for 30 minutes at 25° C. with shaking at 850 rpm on ThermoMixer C shaker (Eppendorf). After 30 min incubation, 6 μL of the MB Block buffer (50 mM D-Biotin in 50 mM Tris-HCl, pH 8, 0.01% Tween) was added to each well of the plate and plates were further incubated for 2 min with shaking. Plates were then washed with 175 μL of the Assay Buffer, wash cycle of 1 min shaking on the ThermoMixer C at 850 rpm followed by separation on the magnet for 30 seconds. After wash solution was removed, beads were resuspended in 175 μL of Assay Buffer and stored at −20° C. until use.

### Catch-2 Bead Preparation.

Before the start of the robotic processing of the assay, 10 mg/mL bead slurry of MyOne Streptavidin C1 beads (Dynabeads, part number 35002D, Thermo Scientific) used for Catch-2 step of the multiplex aptamer assay was washed in bulk once the MB Prep buffer (10 mM Tris-HCl, pH8, 1 mM EDTA, 0.4% SDS) for 5 min followed by two washes with Assay Buffer. After the last wash, beads were resuspended at 10 mg/mL concentration and 75 μL of bead slurry was dispensed into each well of the Catch-2 plate. At the beginning of the assay, Catch-2 plate was placed in the aluminum adapter and placed in the appropriate position on the Fluent deck.

### Sample Binding Step.

Catch-0 plates prepared by immobilizing the aptamer mixes on the Streptavidin Magnetic Sepharose beads as described above. Frozen plates were thawed for 30 min at 25° C. and were washed once with 175 μL of Assay Buffer. 100 μL of each sample dilution (20%, 0.5% and 0.005%) were added to the plates containing beads with three different aptamer master mixes (Dil1, Dil2 and Dil3, respectively). Catch-0 plates were then sealed with aluminum foil seals (Microseal ‘F’ Foil, Bio-Rad) and placed in the 4-plate rotating shakers (PHMP-4, Grant Bio) set at 850 rpm, 28° C. Sample binding step was performed for 3.5 hours.

### Multiplex Aptamer Assay Processing on Fluent Robot.

After sample binding step was completed, Catch-0 plates were placed into aluminum plate adapters and placed on the robot deck. Magnetic bead wash steps were performed using a temperature-controlled plate. For all robotic processing steps, the plates were set at 25° C. temperature except for Catch-2 washes as described below. Plates were washed 4 times with 175 of Assay Buffer, each wash cycle was programmed to shake the plates at 1000 rpm for at least 1 min followed by separation of the magnetic beads for at least 30 seconds before buffer aspiration. During the last wash cycle, the Tag reagent was prepared by diluting 100×Tag reagent (EZ-Link NHS-PEG₄-Biotin, part number 21363, Thermo, 100 mM solution prepared in anhydrous DMSO) 1:100 in the Assay Buffer and poured in the trough on the robot deck. 100 of Tag reagent was added to each of the wells in the plates and incubated with shaking at 1200 rpm for 5 min to biotinylate proteins captured on the bead surface. Biotinylation reactions were quenched by addition of 175 μL of Quench buffer (20 mM glycine in Assay Buffer) to each well. Plates were incubated static for 3 min then washed 4 times with 175 μL of Assay Buffer, washes were performed under the same conditions as described above.

Photo-Cleavage and Kinetic Challenge.

After the last wash of the plates, 90 μL of Photocleavage buffer (2 μM of a oligonucleotide competitor in Assay Buffer; the competitor oligonucleotide (also referred to as Z-Block, has the nucleotide sequence of 5′-(AC-Bn-Bn)₇-AC-3′, where Bn indicates a 5-position benzyl-substituted deoxyuridine residue) was added to each well of the plates. The plates were moved to a photocleavage substation on the Fluent deck. The substation consists of the BlackRay light source (UVP XX-Series Bench Lamps, 365 nm) and three Bioshake 3000-T shakers (Q Instruments). Plates were irradiated for 20 min minutes with shaking at 1000 rpm.

### Catch-2 Bead Capture.

At the end of the photocleavage process, the buffer was removed from Catch-2 plate via magnetic separation, plate was washed once with 100 μL of Assay Buffer. Photo-cleaved eluate containing aptamer-protein complexes was removed from each Catch-0 plate starting with the dilution 3 plate. All 90 μL of the solution was first transferred to the Catch-1 Eluate plate positioned on the shaker with raised magnets to trap any Steptavidin Magnetic Sepharose beads which might have been aspirated. After that, solution was transferred to the Catch-2 plate and the plate was incubated for 3 min with shaking at 1400 rpm at 25° C. After the incubation for 3 min, the magnetic beads were separated for 90 seconds, solution removed from the plate and photocleaved Dil2 plate solution was added to plate. Following identical process, the solution from Dil1 plate was added and incubated for 3 min. At the end of the 3 min incubation, 6 μL of the MB Block buffer was added to the magnetic bead suspension and beads were incubated for 2 min with shaking at 1200 rpm at 25° C. After this incubation, the plate was transferred to a different shaker which was preset to 38° C. temperature. Magnetic beads were separated for 2 minutes before removing the solution. Then, the Catch-2 plate was washed 4 times with 175 of MB Wash buffer (20% glycerol in Assay Buffer), each wash cycle was programmed to shake the beads at 1200 rpm for 1 min and allow the beads to partition on the magnet for 3.5 minutes. During the last bead separation step, the shaker temperature was set to 25° C. Then beads were washed once with 175 μL of Assay Buffer. For this wash step, beads were shaken at 1200 rpm for 1 min and then allowed to separate on the magnet for 2 minutes. Following the wash step, aptamers were eluted from the purified aptamer-protein complexes using Elution buffer (1.8 M NaClO₄, 40 mM PIPES, pH 6.8, 1 mM EDTA, 0.05% Triton X-100). Elution was done using 75 of Elution buffer for 10 min at 25° C. shaking beads at 1250 rpm. 70 μL of the eluate was transferred to the Archive plate and separated on the magnet to partition any magnetic beads which might have been aspirated. 10 μL of the eluted material was transferred to the black half-area plate, diluted 1:5 in the Assay Buffer and used to measure the Cy3 fluorescence signals which are monitored as internal assay QC. 20 μL of the eluted material was transferred to the plate containing 5 μL of the Hybridization Blocking solution (Oligo aCGH/ChIP-on-chip Hybridization Kit, Large Volume, Agilent Technologies 5188-5380, containing a spike of Cyanine 3-labeled DNA sequence complementary to the corner marker probes on Agilent arrays). This plate was removed from the robot deck and further processed for hybridization (see below). Archive plate with the remaining eluted solution was heat-sealed using aluminum foil and stored at −20° C.

### Hybridization.

25 μL of 2× Agilent Hybridization buffer (Oligo aCGH/ChIP-on-chip Hybridization Kit, Agilent Technologies, part number 5188-5380) was manually pipetted to the each well of the plate containing the eluted samples and blocking buffer. 40 μL of this solution was manually pipetted into each “well” of the hybridization gasket slide (Hybridization Gasket Slide—8 microarrays per slide format, Agilent Technologies). Custom SurePrint G3 8x60k Agilent microarray slides containing 10 probes per array complementary to each aptamer were placed onto the gasket slides according to the manufacturer's protocol. Each assembly (Hybridization Chamber Kit—SureHyb enabled, Agilent Technologies) was tightly clamped and loaded into a hybridization oven for 19 hours at 55° C. rotating at 20 rpm.

Post-Hybridization Washing.

Slide washing was performed using Little Dipper Processor (model 650C, Scigene). Approximately 700 mL of Wash Buffer 1 (Oligo aCGH/ChIP-on-chip Wash Buffer 1, Agilent Technologies) was poured into large glass staining dish and used to separate microarray slides from the gasket slides. Once disassembled, the slides were quickly transferred into a slide rack in a bath containing Wash Buffer 1 on the Little Dipper. The slides were washed for five minutes in Wash Buffer 1 with mixing via magnetic stir bar. The slide rack was then transferred to the bath with 37° C. Wash Buffer 2 (Oligo aCGH/ChIP-onchip Wash Buffer 2, Agilent Technologies) and allowed to incubate for five minutes with stirring. The slide rack was slowly removed from the second bath and then transferred to a bath containing acetonitrile and incubated for five minutes with stirring.

### Microarray Imaging.

The microarray slides were imaged with a microarray scanner (Agilent G4900DA Microarray Scanner System, Agilent Technologies) in the Cyanine 3-channel at 3 μm resolution at 100% PMT setting and the 20-bit option enabled. The resulting tiff images were processed using Agilent Feature Extraction software (version 10.7.3.1 or higher) with the GE1_1200_Jun14 protocol.

### Certain Biological Sample Collection and Preparation

In some embodiments, biological samples are prepared by performing the sample collection, buffer exchange, and/or protein standardization described below.

### Urine Sample Collection and Preparation

Urine samples were collected from individuals via mid-stream capture in a tube and then immediately stored in at −80° C. freezer. Frozen urine samples were processed as follows:

Buffer Exchange: Samples were thawed in a 37° C. water bath for about 15 minutes to assure all cryo-precipitates dissolve. The thawed samples were removed from the water bath and placed in biological safety cabinet. The caps were removed and the contents mixed by pipetting up-and-down three times with a 200 μL multichannel pipette. The samples were aliquoted at 216 each into a new tube. 1M Tris (Tris(hydroxymethyl)aminomethane) pH=8, was added to each aliquot of thawed/mixed urine using a 20 μL single channel pipette such that final Tris concentration was 40 mM (4%). The Tris-treated aliquots were mixed by gently pipetting up-and-down three times with a 200 μL it multichannel pipette. A 15 μL of Tris-treated urine sample was transferred to a corresponding well in a PCR plate (96-well PCR plate (Fisher Scientific #NC0508659)). This 15 μL it aliquot was stored at −80° C. until protein concentration quantification was performed with a NanoOrange® kit, which comprises a merocyanine dye. (See, e.g., Jones et al. “Development and Characterization of the NanoOrange® Protein Quantitation Assay: A Fluorescence-Based Assay of Proteins in Solution.” BioTechniques. 34(4): 850-4 (2003).

The aliquots were buffer-exchange over two Zeba™ 7 kDa MWCO Spin Desalting Plates (Thermo Scientific #89808) or two Harvard G25 1.5 kDa MWCO 96-Well SpinColumns™ (Harvard Apparatus #74-5652) according to the respective manufacturer's instructions. For the Zeba™ desalting plates, refer to the ‘Procedure for Buffer-Exchange’ section of the manufacturer's protocol. In brief, the Zeba™ Spin Desalting Plates were incubated until room temperature was reached. The assemblies were centrifuged with a 96-well plate-carrier rotor at 1,000×g for 2 minutes to remove the storage buffer. The flow-through was discarded. A 250 μL volume of buffer was added to the top of the resin beds. Again, the plates were centrifuged at 1,000×g for 2 minutes and the flow-through was discarded. This step was repeated three additional times. The wash plate was rinsed three times with deionized water, dried and saved for future use. Each desalting plate was stacked on top of a sample collection plate, thus aligning the alphanumeric indices on the plates. A 100 μL sample was applied to the center of the resin bed of the first desalting plate. Another 100 μL sample of the same sample was applied to the center of the resin bed of the second desalting plate. The place was centrifuged at 1,000×g for 2 minutes and the processed sample was collected. A 200 μL it multichannel pipette was used to transfer 100 μL of each Tris-treated/desalted sample to each of two matrix racks, and sample orientation was maintained. The matrix tubes were re-capped and now contained 100 μL of Tris-treated/desalted sample. The aliquot tubes were transferred to dry ice and stored at −80° C. For the Harvard G25 96-Well SpinColumns™ refer to the ‘For Gel Filtration/Ion Exchange Columns’ section of the manufacturer's protocol. Briefly, the 96-Well SpinColumns™ were placed into one of the collection tubes, and 200 μL of buffer was added into all open wells. The tubes were incubated for 15 minutes to allow for hydration. The plates were centrifuged for 2 minutes at 2,000×g. The 96-Well SpinColumns™ were removed from the collection plates and blotted to dry any moisture on the exterior of the column. A 100 μL volume of the sample was transferred to the top of the well of the first 96-Well Spin Column plate. Another 100 μL volume of the same sample was added to the center of the well of the second 96-Well Spin Column plate. Each column plate was added to a new collection plate and spun for 2 minutes at 2,000×g. 100 μL of each Tris-treated/desalted sample was transferred to each of two matrix racks. The matrix tubes were re-capped and now contain 100 μL of Tris-treated/desalted sample. The racks of completed aliquot tubes were transferred to dry ice and stored at −80° C.

Protein Standardization to 15 μg/mL Total Protein:

Buffer-exchanged urine samples were standardized to a total protein concentration of 15 μg/mL in 120 μL as follows: The buffer-exchanged urine specimen was thawed in a 28-37° C. water bath for 10 minutes. The volume of the buffer-exchanged specimen needed was calculated by the following equation: 0.12 mL×15 μg/mL/Urinary [Protein] (μg/mL)=V*(volume of urine sample). If by this equation V* was greater than 0.108 mL then V* was adjusted to be 0.108 mL. 1× Assay Buffer was added to a tube by the following equation: 0.108 mL−V*=Volume of Assay Buffer. A 0.012 mL volume of 1× Assay Buffer containing 50 μM Z-block (10× Z-block) was added. The sample was mixed gently. A 100 μL volume was transferred to a single well of a 96-well Catch-0 plate of the aptamer-based assay. All three plasma Master Mixes (Dil1, Di12, and Di13) were combined into each well.

### Kits

Any combination of the analytes described herein can be detected using a suitable kit, such as for use in performing the methods disclosed herein. Furthermore, any kit can contain one or more detectable labels as described herein, such as a fluorescent moiety, etc.

In the foregoing description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

Some portions of the detailed description have been presented in terms of steps leading to one or more desired results. Generally, such steps are those requiring physical manipulations of physical quantities. Usually, though not necessarily, these quantities take the form of electrical or magnetic signals capable of being stored, transferred, combined, compared, and otherwise manipulated. It has proven convenient at times, for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “computing”, “comparing”, “applying”, “creating”, “ranking,” “classifying,” or the like, refer to the actions and processes of a specialized computer system, or similar specialized electronic computing device, that manipulates and transforms data represented as physical (e.g., electronic) quantities within the computer system's registers and memories into other data similarly represented as physical quantities within the computer system memories or registers or other such information storage, transmission or display devices.

Certain examples of the present disclosure also relate to an apparatus for performing the operations herein. This apparatus may be constructed for the intended purposes, or it may comprise specialized computer hardware and/or specialized computer programs selectively installed, activated, or configured to perform the intended purposes. Such computer programs may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions.

It is to be understood that the above description is intended to be illustrative, and not restrictive. Many other examples will be apparent to those of skill in the art upon reading and understanding the above description. The scope of the disclosure should, therefore, be determined with reference to the appended claims, along with the full scope of equivalents to which such claims are entitled.

### EXAMPLES

The following examples are provided to illustrate certain particular features and/or embodiments. These examples should not be construed to limit the disclosure to the particular features or embodiments described.

### Example 1: Total Protein and Composition Variability of Urine

To confirm inter-sample variability in urine samples, the NanoOrange® protein quantitation method (Invitrogen; Molecular Probes NanoOrange® Protein Quantitation Kit; Catalog #N6666) was used with three different urine collections, and resulted in a wide range of protein concentrations from the spot urine specimens. Group 1 consisted of 46 volunteers, Group 2 consisted of 170 volunteers and Group 3 consisted of 183 volunteers. Generally, urine samples were collected from individuals via mid-stream captured in a tube and then immediately stored in at −80° C. freezer.

Protein concentrations for the three different urine collection groups ranged from about 4 μg/ml to about 4,423 μg/mL, resulting in an approximately 1,000-fold difference between the most dilute and most concentrated samples. Median (interquartile range) protein concentrations for each collection are 46.5 (23-74.5) μg/mL for Group 1 volunteers, 57.5 (27.2-89.8) μg/mL for Group 2 subjects, and 75.0 (51-114) μg/mL for Group 3 subjects.

Generally, the methodology used to determine the urinary total protein concentration using the NanoOrange kit was as follows: A 1× protein quantitation diluent was made by diluting the 10× concentrated NanoOrange® protein quantitation diluent 10-fold in distilled water. For each 96-well plate, 15 mL of 1× protein quantitation diluent was required. 1.5 mL NanoOrange® protein quantitation diluent was added to 13.5 mL of distilled or deionized water. For each 96-well plate, 13 mL of 1.04× NanoOrange® reagent working solution was prepared. The NanoOrange® protein quantitation reagent (Component A) was diluted 481-fold into the 1× protein quantitation diluent. In a foil-wrapped tube, 0.027 mL NanoOrange® protein quantitation reagent (Component A) was added to 13 mL 1× protein quantitation diluent. The 1× NanoOrange® working solution was protected from light to prevent photo degradation of the NanoOrange® dye. The NanoOrange® Kit includes a 2 mg/mL sample of BSA (Component B) that was used to prepare a standard curve. The standard curve was generated at 25× and was covered in a FINAL range of from 10.25-0.402 μg/mL. An initial 25× stock solution of BSA (10.25 μg/mL FINAL) in 1× protein quantitation diluent final was prepared. 39 μL of 10× NanoOrange® protein quantitation diluent (as supplied by the manufacturer) and 50 μL of 2 mg/mL BSA was added to 302 μL distilled water. Eight 1.5× serial dilutions of the 25×BSA solution in 1× NanoOrange® protein quantitation diluent was prepared. 200 μL previous standard was added to the 100 μL 1× NanoOrange® protein quantitation diluent. 25×QC #1 at 205 μg/mL (8.2 μg/mL final) in 1× protein quantitation diluent was prepared. 48.8 μL of 10× NanoOrange® protein quantitation diluent (as supplied by the manufacturer) and 50 μL of 2 mg/mL BSA was added to 389.2 μL distilled water. 25×QC #2 at 132.5 μg/mL (5.3 μg/mL final) in 1× protein quantitation diluent was prepared. 226 μL QC #1 was added to 123.7 μL of 1× NanoOrange® protein quantitation diluent. 25×QC #3 at 30 μg/mL (1.2 μg/mL final) in 1× protein quantitation diluent was prepared. 79 μL QC #2 was added to 269.9 μL of 1× NanoOrange® protein quantitation diluent. Each Tris-Treated urine sample (see processing note below) 25× was diluted into 1.04× NanoOrange® reagent working solution. In a non-skirted PCR plate (Abgene #AB-0600-L), 6 μL raw urine sample was added to 144 μL 1.04× NanoOrange® reagent working solution. Each QC sample 25×, was diluted, in duplicate, into 1.04× NanoOrange® reagent working solution. In a non-skirted PCR plate (Abgene #AB-0600-L), 6 μL QC sample was added, in duplicate, to 144 μL 1.04× NanoOrange® reagent working solution. Each Standard Curve sample 25× was diluted into 1.04× NanoOrange® reagent working solution. In a non-skirted PCR plate (Abgene #AB-0600-L), 6 μL Standard Curve sample was added to 144 μL 1.04× NanoOrange® reagent working solution. Two “blanks” were included.

In a non-skirted PCR plate (Abgene #AB-0600-L), 6 μL 1× NanoOrange® protein quantitation diluent was added to 144 μL 1.04× NanoOrange® reagent working solution. All wells containing sample with MicroAmp Optical 8-cap strips were capped (Applied BioSystems #N8010560). Samples were incubated at 95° C. for 10 minutes in a thermocycler with heated lid, and protected from light and then cooled to room temperature for 30 minutes, either in the thermocycler or on the benchtop, and protected from light. A multichannel pipette was used to transfer 100 μL of each sample to a black-walled, clear bottomed, half area 96-well plate (Corning #3880). The fluorescence was measured in a SpectraMax M5 (or similar instrument) at 470 nm. The parameters of SpectraMax were set to: Fluorescence Endpoint; Top Read: 470 nm excitation/590 nm emission/570 cutoff; Automix: off; Calibrate: on; PMT: Auto; Settle Time: off; Column Priority; Speed: Normal; Reads/Well: 6.

The protein concentration of unknown samples was determined by first subtracting the fluorescence value of the reagent blanks from that of all samples, including QC's and Standards. A standard curve with a 4-parameter fit was used to determine the protein concentrations of the unknown samples and QC's. The % CV (<15%) and Accuracy (<15%) of the duplicate QC samples was calculated to confirm the plate passes.

Further, the effect of Tris-HCl (pH=8) addition on the NanoOrange protein quantitation assay was evaluated on seventeen independent urine specimens. Fresh-frozen urine aliquots were thawed in a 37° C. water bath for 15 minutes, centrifuged at 14,000 g for 10 minutes and clarified supernatants transferred to a fresh tube. Six microliter aliquots were removed from the clarified supernatants for protein quantitation in the NanoOrange assay. Tris pH 8.0 (1M) was then added to the remaining 44 μL supernatants to a final concentration of 40 mM. Six microliters of these Tris-treated samples were similarly run in the NanoOrange assay and protein concentrations compared to their untreated counterparts.

Briefly, 6 μL of urine were added to 144 μL of 1× NanoOrange working solution in a PCR plate. Samples were capped and heated to 95° C. for 10 min in a thermocycler and allowed to cool on the benchtop for 30 min, protected from light. 100 μL of heat-cooled sample was then transferred to a clear-bottom/black-walled half-area UV plate and fluorescence measured using 470 nm excitation/590 nm emission wavelengths. Protein concentrations were determined using a standard curve included on each plate.

This example confirmed that urine specimens from both multiple subjects and from a single subject over multiple collection times exhibit variability in total protein measurements. Further, this example showed that the composition and characteristics (e.g., pH and salt content) of urine samples can vary subject to subject.

### Example 2: Analyte Measurements are Sensitive to Different Concentrations of Salts and Urea in a Proteomic Assay

This example shows that analyte measurements in a proteomic assay are sensitive to changes in the concentrations of salts and urea, both of which are components of urine.

The effects of various concentrations of urea, NaCl and/or MgCl₂ on an aptamer-based proteomic assay were measured. The control (or “truth standard”) formulation used in this Example was SB17 assay buffer. SB17 is an Assay Buffer that was made by combining 102 mM NaCl, 5 mM KCl, 5 mM MgCl₂, 40 mM HEPES, 1 mM EDTA, and 0.1% Tween-20 (v/v), at pH 7.5. Formulations of the SB17 buffer having variable salt and/or urea concentrations were made (see Formulations 1 through 28 in Table 3) and spiked with one protein selected from the 201 recombinant proteins listed in Table 2 at a specified protein concentration.

TABLE 2
Recombinant Proteins
Recombinant Proteins

| | | | | |
|---|---|---|---|---|
| TIMP-3 | ADAMTS-5 | CAIII | Soggy-1 | RET |
| IL-12 Rb1 | HB-EGF | VEGF | CHST2 | F7 |
| GH | Cytochrome | ALK-1 | DSC3 | Kallikrein 11 |
| receptor | c | | | |
| NXPH1 | JAM-B | Cadherin-5 | TRY3 | NRX1B |
| GFRa-2 | BMPR1A | BCMA | MIP-1a | TCCR |
| IL-20 Ra | TIMP-1 | Contactin-5 | CNTN2 | IL-8 |
| LY9 | RTN4 | TSP2 | ANGPT-4 | LY86 |
| CD110 | IL-17 | FN1.3 | SREC-I | Cystatin-S |
| AMNLS | IL-2 sRa | Eck | ARSB | CD30 |
| PRLR | LIGHT | GP114 | Lymphotactin | Lymphotoxin |
| | | | | a2/b1 |
| AHSG | RGMB | Ubiquitin+1 | ST4S6 | SORC2 |
| KIRR3 | TWEAKR | M-CSF R | FGF-8A | Notch-3 |
| OX40 | RANTES | TGF-b R III | b-NGF | BCL2-like |
| Ligand | | | | 1 protein |
| P-Selectin | EphB4 | CTLA-4 | Chymase | ILT-2 |
| LRRT1 | EphA5 | IL-5 Ra | Cadherin-12 | GPNMB |
| GPVI | UNC5H3 | FLRT1 | Protease nexin I | ECM1 |
| GRN | DR6 | DC-SIGN | VCAM-1 | CD23 |
| Siglec-9 | IGF-I sR | DC-SIGNR | IL-17 RC | DPP2 |
| Eotaxin-2 | CTACK | sICAM-1 | CATZ | C1s |
| HTRA2 | Siglec-6 | PDGF-AA | FUT3 | ENTP3 |
| LTBR | FGF23 | JAG1 | MMP-12 | XPNPEP1 |
| HPLN1 | IL-4 sR | MMP-13 | DKK1 | MMP-9 |
| B7-2 | EphA1 | CD27 | CHST6 | CD39 |
| Ephrin-A4 | CX3CL-1 | ART | Kallikrein 7 | ADAMTS-4 |
| BCAM | GP1BA | ANGL3 | CFC1 | Heparin |
| | | | | cofactor II |
| pTEN | I-309 | IL-17 RD | DHH | ASAH2 |
| EDAR | ASGR1 | IL-11 RA | GPC5 | TPSG1 |
| SLIK5 | ICOS | MMP-3 | Galectin-8 | C2 |
| FCG3B | MICA | sTie-2 | IGFBP-4 | CATC |
| TSP4 | CD226 | IGFBP-6 | AMHR2 | Enterokinase |
| NCAM-L1 | MMP-2 | ESAM | Adiponectin | MMP-14 |
| IL-1Rrp2 | ANGPT-2 | SLPI | MCP-2 | dopa |
| | | | | decarboxylase |
| IL22RA1 | NLGNX | RBP | MEPE | ATS15 |
| IFN-g R1 | FCN2 | Decorin | Kallistatin | PAFAH |
| SCF sR | annexin VI | Activin AB | VEGF-C | IDUA |
| NKp44 | MICB | BMPER | Aminoacylase-1 | CNDP1 |
| Cystatin | ULBP-1 | AREG | Ubiquitin | LIMP II |
| M | | | | |
| TSG-6 | Met | CD36 | Renin | Angiopoietin-1 |
| | | ANTIGEN | | |
| IL-1 R | CLC7A | granzyme A | IL-3 Ra | IL-23 |
| AcP | | | | |
| EphB6 | BOC | NRX3B | Caspase-2 | CATE |
| MCP-3 | | | | |

TABLE 3
The Components and their Respective Concentrations Combined to Make the Assay Buffer
Formulations Used in the Aptamer-based Assay

| | Formulation Components Combined with Concentrations (pH~7.5) | | | | | | |
|---|---|---|---|---|---|---|---|
| Formulation | NaCl | MgCl₂ | Urea | KCl | HEPES | EDTA | Tween20 |
| 1X SB17 | 102 mM | 5 mM | 0 | 5 mM | 40 mM | 1 mM | 0.1% |

Increasing NaCl Formulations

| | | | | | | | |
|---|---|---|---|---|---|---|---|
| Formulation 1 | 112 mM | 5 mM | 0 | 5 mM | 40 mM | 1 mM | 0.1% |
| Formulation 2 | 122 mM | 5 mM | 0 | 5 mM | 40 mM | 1 mM | 0.1% |
| Formulation 3 | 142 mM | 5 mM | 0 | 5 mM | 40 mM | 1 mM | 0.1% |
| Formulation 4 | 182 mM | 5 mM | 0 | 5 mM | 40 mM | 1 mM | 0.1% |
| Formulation 5 | 262 mM | 5 mM | 0 | 5 mM | 40 mM | 1 mM | 0.1% |
| Formulation 6 | 342 mM | 5 mM | 0 | 5 mM | 40 mM | 1 mM | 0.1% |
| Formulation 7 | 422 mM | 5 mM | 0 | 5 mM | 40 mM | 1 mM | 0.1% |

Increasing MgCl₂ Formulations

| | | | | | | | |
|---|---|---|---|---|---|---|---|
| Formulation 8 | 102 mM | 5.25 mM | 0 | 5 mM | 40 mM | 1 mM | 0.1% |
| Formulation 9 | 102 mM | 5.5 mM | 0 | 5 mM | 40 mM | 1 mM | 0.1% |
| Formulation 10 | 102 mM | 6 mM | 0 | 5 mM | 40 mM | 1 mM | 0.1% |
| Formulation 11 | 102 mM | 7 mM | 0 | 5 mM | 40 mM | 1 mM | 0.1% |
| Formulation 12 | 102 mM | 9 mM | 0 | 5 mM | 40 mM | 1 mM | 0.1% |
| Formulation 13 | 102 mM | 11 mM | 0 | 5 mM | 40 mM | 1 mM | 0.1% |
| Formulation 14 | 102 mM | 13 mM | 0 | 5 mM | 40 mM | 1 mM | 0.1% |

Increasing Urea Formulations

| | | | | | | | |
|---|---|---|---|---|---|---|---|
| Formulation 15 | 102 mM | 5.25 mM | 10 mM | 5 mM | 40 mM | 1 mM | 0.1% |
| Formulation 16 | 102 mM | 5.25 mM | 20 mM | 5 mM | 40 mM | 1 mM | 0.1% |
| Formulation 17 | 102 mM | 5.25 mM | 40 mM | 5 mM | 40 mM | 1 mM | 0.1% |
| Formulation 18 | 102 mM | 5.25 mM | 80 mM | 5 mM | 40 mM | 1 mM | 0.1% |
| Formulation 19 | 102 mM | 5.25 mM | 160 mM | 5 mM | 40 mM | 1 mM | 0.1% |
| Formulation 20 | 102 mM | 5.25 mM | 240 mM | 5 mM | 40 mM | 1 mM | 0.1% |
| Formulation 21 | 102 mM | 5.25 mM | 320 mM | 5 mM | 40 mM | 1 mM | 0.1% |

Increasing Combined Formulations

| | | | | | | | |
|---|---|---|---|---|---|---|---|
| Formulation 22 | 112 mM | 5.25 mM | 10 mM | 5 mM | 40 mM | 1 mM | 0.1% |
| Formulation 23 | 122 mM | 5.5 mM | 20 mM | 5 mM | 40 mM | 1 mM | 0.1% |
| Formulation 24 | 142 mM | 6 mM | 40 mM | 5 mM | 40 mM | 1 mM | 0.1% |
| Formulation 25 | 182 mM | 7 mM | 80 mM | 5 mM | 40 mM | 1 mM | 0.1% |
| Formulation 26 | 262 mM | 9 mM | 160 mM | 5 mM | 40 mM | 1 mM | 0.1% |
| Formulation 27 | 342 mM | 11 mM | 240 mM | 5 mM | 40 mM | 1 mM | 0.1% |
| Formulation 28 | 422 mM | 13 mM | 320 mM | 5 mM | 40 mM | 1 mM | 0.1% |

As shown in Table 3, the final concentrations of NaCl in the formulations varied from about 100 mM to about 420 mM, more specifically concentrations of NaCl were 112 mM, 122 mM, 142 mM, 182 mM, 262 mM, 342 mM and 422 mM. The final concentrations of MgCl₂ in the formulations varied from about 5 mM to about 13 mM, more specifically concentrations of MgCl₂ were 5.25 mM, 5.5 mM, 6 mM, 7 mM, 9 mM, 11 mM and 13 mM. The final concentrations of urea in the formulations varied from about 0 mM to about 320 mM, more specifically concentrations of urea were 0 mM, 20 mM, 20 mM, 40 mM, 80 mM, 160 mM, 240 mM and 320 mM.

The concentrations of the 201 recombinant proteins in the SB17 buffer and Formulations 1 through 28 were measured with an Aptamer Assay Method.

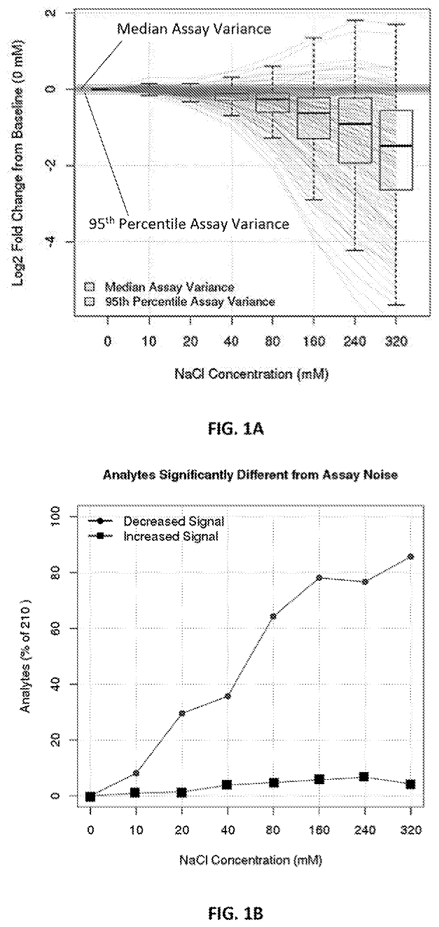

The data for the effect of increasing concentration of NaCl on the measurement of the 201 recombinant proteins in the aptamer-based assay are shown in FIG. 1. The Log₂-fold change in RFU (relative fluorescence units), which corresponds to the relative level of protein present in the assay, for the 201 protein analytes compared to the control (or baseline comparator measurements) shows that as the concentration of NaCl increased, the measured level of the individual protein analytes by the assay changed. Generally, these data indicate that if two or more samples (e.g., urine samples) having the same concentration of one or more proteins yet different NaCl concentrations were used in the aptamer-based assay, the resulting levels of the one or more proteins from the two or more samples would be different, thus confounding any results related to biomarker discovery and/or a diagnostic.

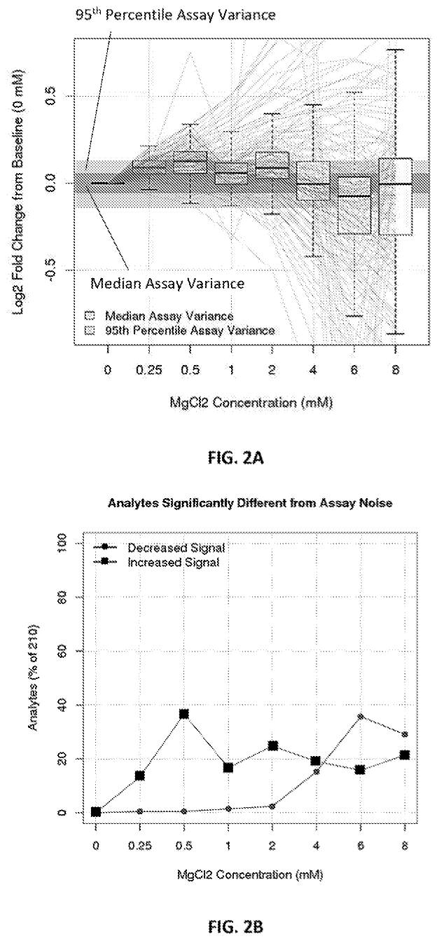

The data for the effect of increasing concentration of MgCl₂ on the measurement of the 201 recombinant proteins in the aptamer-based assay are shown in FIG. 2. The Log 2-fold change in RFU (relative fluorescence units), which corresponds to the relative level of protein present in the assay, for the 201 protein analytes compared to the control (or baseline comparator measurements) shows that as the concentration of MgCl₂ increased, the measured level of the individual protein analytes by the assay changed. Generally, these data indicate that if two or more samples (e.g., urine samples) having the same concentration of one or more proteins yet different MgCl₂ concentrations were used in the aptamer-based assay, the resulting levels of the one or more proteins from the two or more samples would be different.

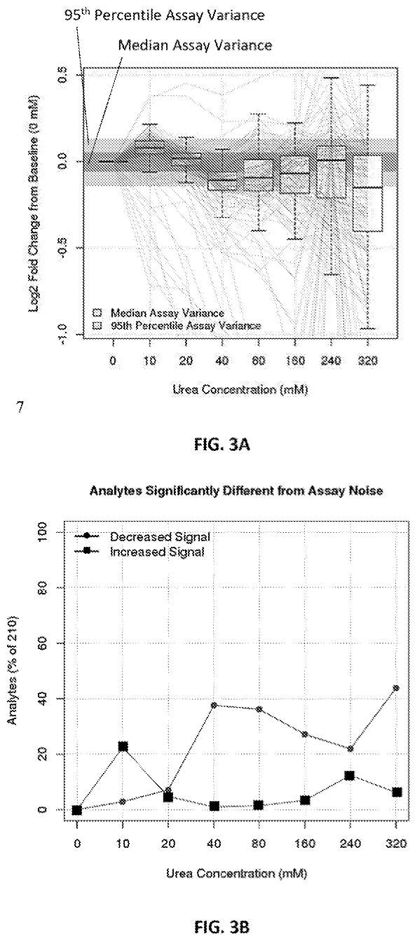

The data for the effect of increasing concentration of urea on the measurement of the 201 recombinant proteins in the aptamer-based assay are shown in FIG. 3. The Log 2-fold change in RFU (relative fluorescence units), which corresponds to the relative level of protein present in the assay, for the 201 protein analytes compared to the control (or baseline comparator measurements) shows that as the concentration of urea increased, the measured level of the individual protein analytes by the assay changed. Generally, these data indicate that if two or more samples (e.g., urine samples) having the same concentration of one or more proteins yet different urea concentrations were used in the aptamer-based assay, the resulting levels of the one or more proteins from the two or more samples would be different.

Collectively, these data indicate that varying concentrations of salts affected the measured level of a protein in an aptamer-based assay. Such variations complicate biomarker discovery and diagnostic tests, particularly for biological samples that are known to have varying salt concentrations among samples (e.g., urine). Accordingly, the varying concentrations of salts must be mitigated in order to compare the level of a protein or set of proteins from biological samples and derive meaningful health related information and/or clinical decisions tools (e.g., disease vs. non-disease).

### Example 3: Use of a Fixed Protein Concentration in Urine to Measure Analytes with an Aptamer-Based Assay

This example shows that the total protein concentration in urine affects the number of analytes that are in the linear range in an aptamer-based assay.

The linear range of the binding curve is the region in which a change in target concentration corresponds to a proportional (i.e. linear) change in relative fluorescent units (RFU) signal (that is the rate of change in concentration is equal to the rate of change in RFU, which is the measured units of a hybridization based readout for an aptamer-based assay). Generally, the preferred concentration or range of concentrations of total protein for any type of sample is defined as the concentration which maximizes the number of analytes in the linear range.

To determine a preferred concentration or range of concentrations of total protein for urine, an 8-point titration was performed with a urine sample having a high protein concentration, for example, 147 μg/mL. As the aptamer assay buffers take up about 20% of the reaction volume, about 80% of the 147 μg/mL concentration, or 118 μg/mL, of the specimen was the starting protein concentration of the sample. A 6-point 1:2 dilution series to 1.8 μg/mL was then performed, with a separate point at 88 μg/mL (or 75% of the starting protein concentration) added for slightly more resolution at higher concentrations. Samples were assayed on the aptamer-based assay described herein. All samples were assayed in singlicate with 20 μM Z-block.

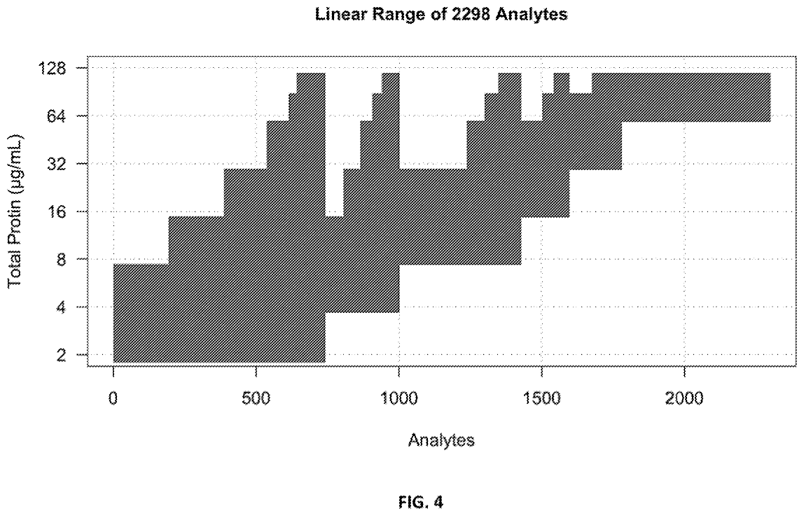

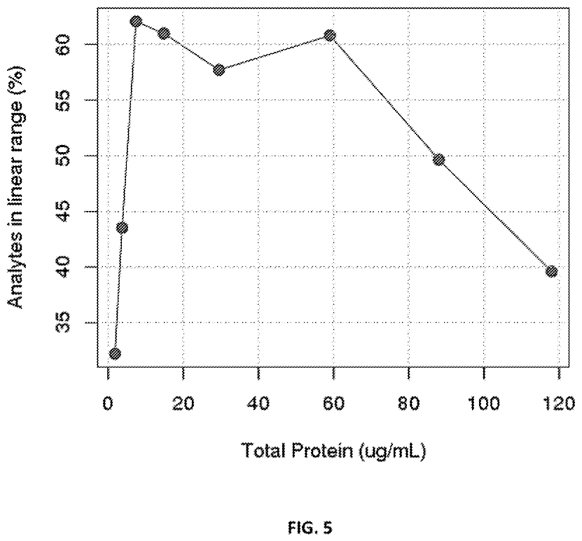

A total of 2,298 analytes were observed to have 3+ point dilution linearity, which the linearity was dependent upon the starting total protein concentration. Linear ranges are shown in FIG. 4. The X-axis identifies the number of analytes (from 0 to 2,298 analytes) and the Y-axis identifies the total protein concentration (from 2 μg/mL to 128 μg/mL). The black bars span the lower and upper limits of the linear range for each analyte as a function of the total protein concentration. While each total protein concentration had analytes that fell within a linear range, over 60% of the analytes had a linear range when the sample was standardized to a total protein concentration of 15 μg/mL. Thus, a linear range for analytes was observed at a total protein concentration of 2, 4, 8, 16, 32, 64 and 128 μg/mL. An alternative way of looking at the same data is shown in FIG. 5.

FIG. 5 shows the percent of the total number of analytes measured (n=2,298) in the linear range as a function of total protein concentration. Starting from 0 μg/mL, a local maximum of analytes in the linear range occurred at about 7 μg/mL and then a slight decrease is observed until about 30 μg/mL. Though an increase at 59 μg/mL total protein concentration was observed, the measured signal (i.e., RFU levels) was relatively low (data not shown), and thus may have been difficult to measure consistently.

Collectively, these data indicate that while a linear range may be obtained for analytes at total protein concentrations ranging from about 2 μg/mL to about 128 μg/mL, the preferred total protein concentration range that provides for the greatest number of analytes in the linear range along with relatively “strong” RFU signals, was from about 2 μg/mL to 50 μg/mL as measured by NanoOrange, and more preferably from about 5 μg/mL to 40 μg/mL and the most preferred was about from 10 μg/mL to 30 μg/mL. More precise total protein concentrations that provide the greatest number of analytes in the linear range were about 7 μg/mL, 10 μg/mL and 15 μg/mL.

Though these data indicate that adjusting the total protein concentration in a sample to a standardized total protein concentration prior to measuring protein analytes in the sample can increase the number of analytes in the linear range, complex matrices, such as urine, will still contain various components, such as salts, that may interfere with the ability to accurately and consistently measure the level of a protein in that sample.

### Example 4: Use of Buffer Exchange on Urine Samples to Measure Analytes with an Aptamer-Based Assay

This example shows that applying a buffer exchange methodology to urine samples affects the number of analytes having a linear range that may be measured by an aptamer-based assay.

A titration was performed on urine samples without buffer exchange (control) and after a buffer exchange using SB17 assay buffer. Buffer exchange was accomplished using a Zeba™ 7 kDa MWCO spin desalting column and following the manufacturer's protocol. The dilution series was 4, 8.1, 16.3, 32.5, 65 and 130 μg/mL total protein for both the control and buffer-exchanged urine samples. The 2,298 analytes in each of the control and the buffer-exchanged samples were individually measured in an aptamer-based assay as outlined in Example 3.

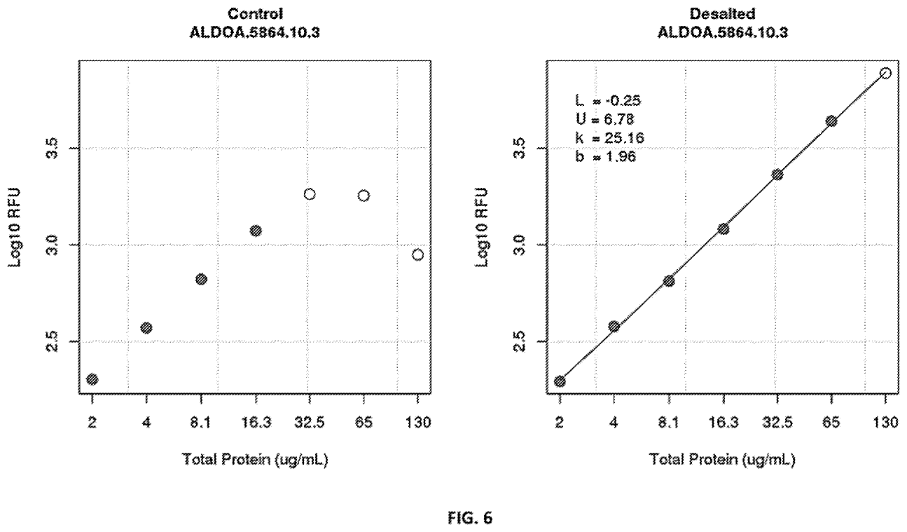

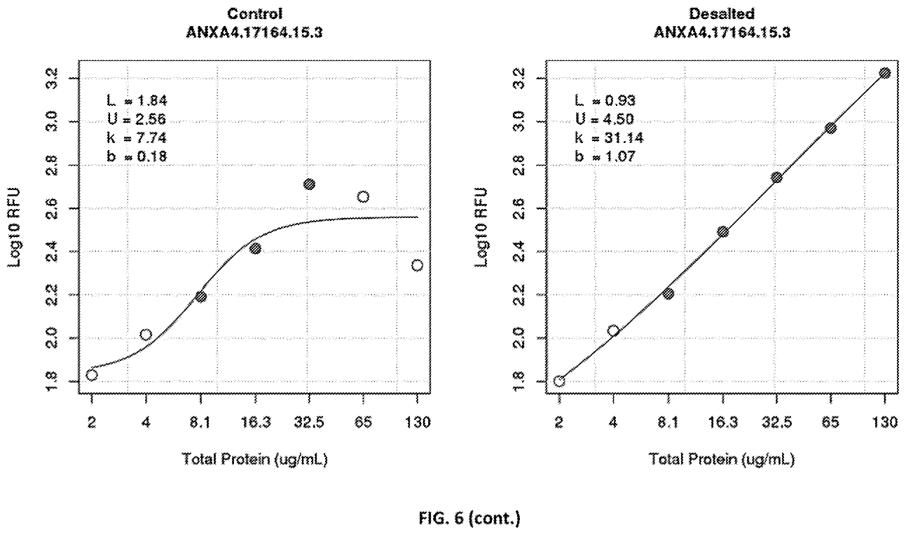

The results of the aptamer-based assay for the total protein titration series for two analytes, ALDOA and ANXA4, are shown in FIG. 6. Buffer-exchanged (identified as “Desalted” in FIG. 6) and non-buffer-exchanged urine samples were plotted with the x-axis being the total protein concentration of the sample, and the y-axis being the Log₁₀ RFU value (the surrogate for the relative level of the protein in the sample). FIG. 6 shows that performing a buffer exchange of a urine sample prior to measuring an analyte in an aptamer-based assay, extended the linear range of the analyte, particularly above about 16 μg/mL total protein.

The other analytes tested showed similar results. These data indicate that higher total protein concentrations, which likely represent a greater urine content (less dilute sample) and thus greater concentration of assay “interfering substances” (e.g., urine salts), affected analyte measurements in the assay. Further, these data indicate that by performing a buffer exchange on the urine sample prior to assaying for the level of analytes in the sample mitigated the impact that these “interfering substances” had on the ability to consistently and reproducibly measure certain analytes in a urine sample.

Thus, buffer exchange of a urine sample prior to measuring the level of an analyte in an aptamer-based assay mitigated the impact of urine “interfering substances”, particularly for urine samples with protein concentration of greater than about 16 μg/mL (e.g., ALDOA protein) and 32 μg/mL (e.g., ANXA4 protein).

### Example 5: Combined Buffer Exchange and Total Protein Concentration Standardization of Urine Samples

This example shows that the combination of applying a buffer exchange and total protein concentration standardization to urine samples improves the reproducibility of measuring analytes and the number of analytes in the sample. This likely will result in a greater ability to perform biomarker discovery and diagnostic or prognostic type assays on individuals using a urine sample with a proteomic assay.

A titration experiment was performed to determine the total protein concentration for buffer-exchanged urine samples that would provide the greatest number of analytes having a linear range. Eleven urine samples from human donors were buffer-exchanged into SB17 buffer using 2 mL Zeba spin desalting columns (400 μL urine load). Titrations were performed by serial 2-fold dilution starting with 80% “urine” while keeping the oligonucleotide competitor (Z-block) concentration at 5 μM. Titration curves for each sample were fitted and the number of analytes in the linear range in total protein space as measured by NanoOrange, as well as those analytes above and below the linear range, were calculated and plotted.

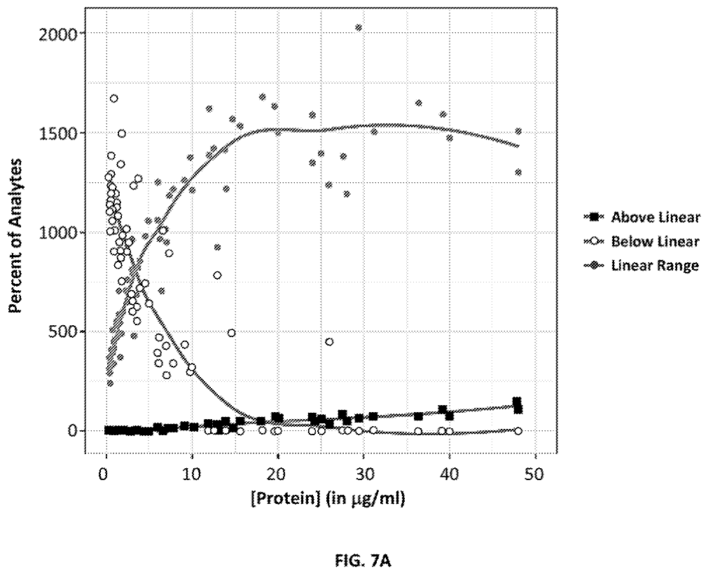

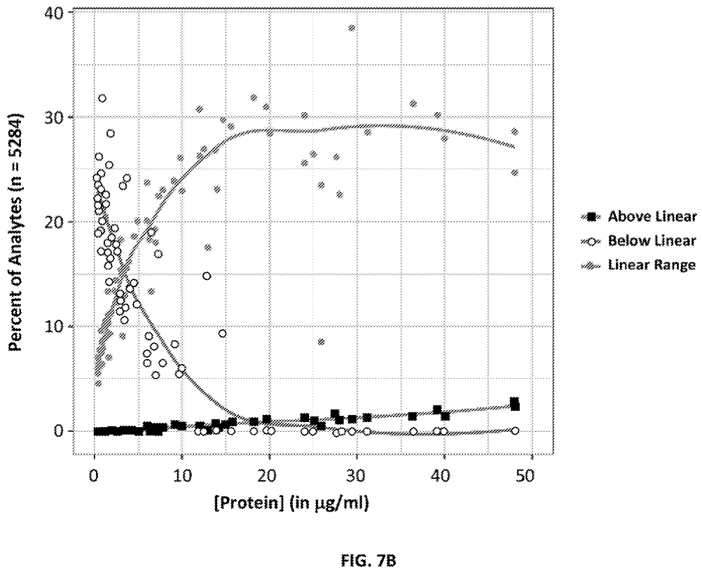

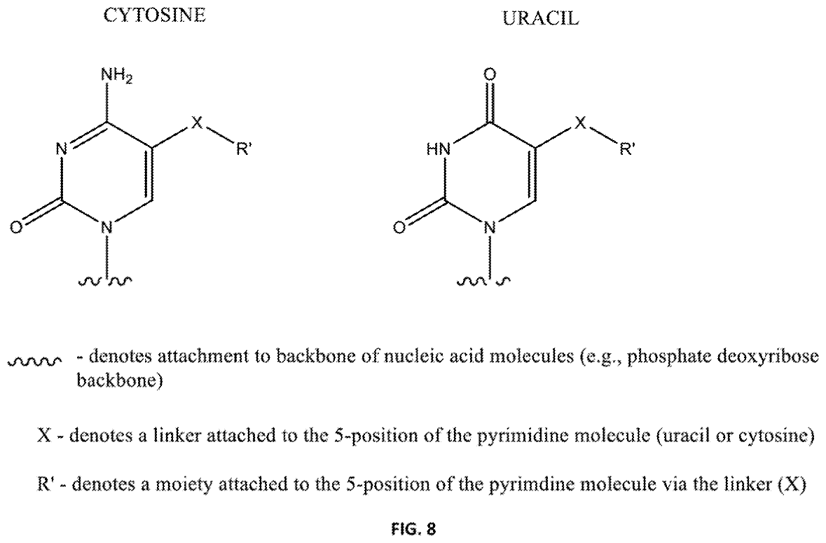

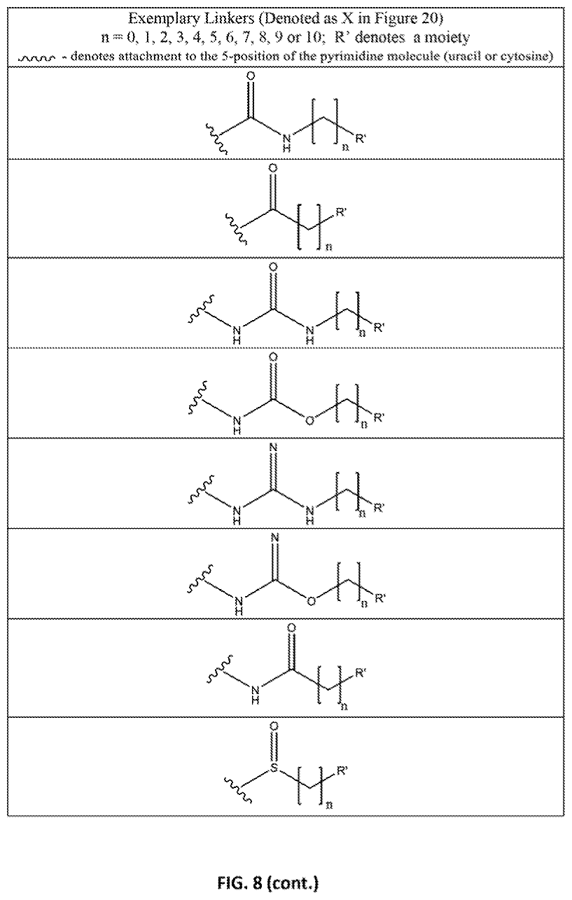

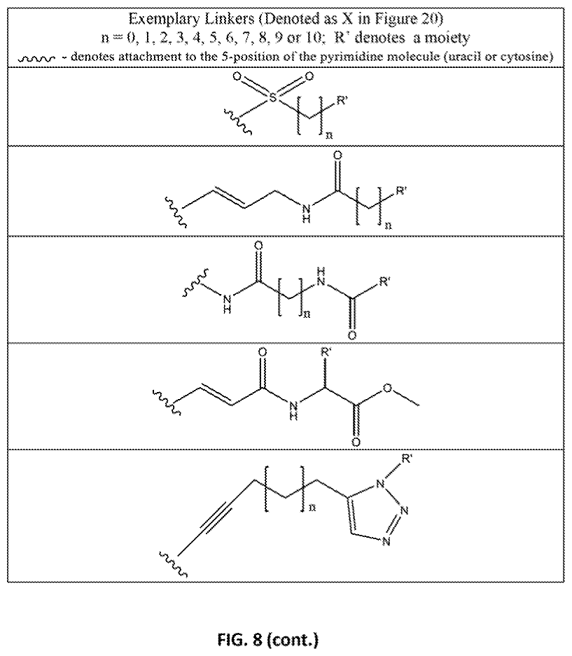

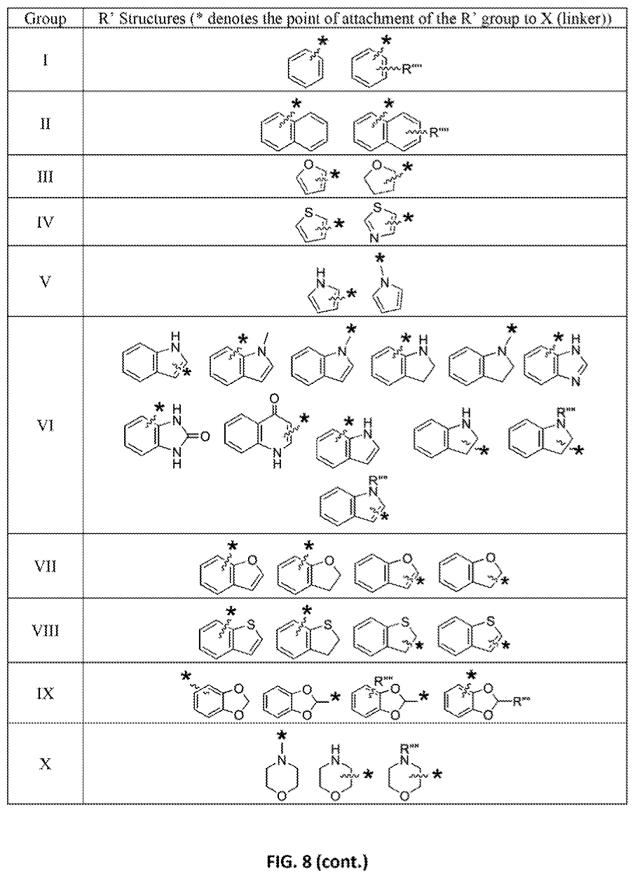

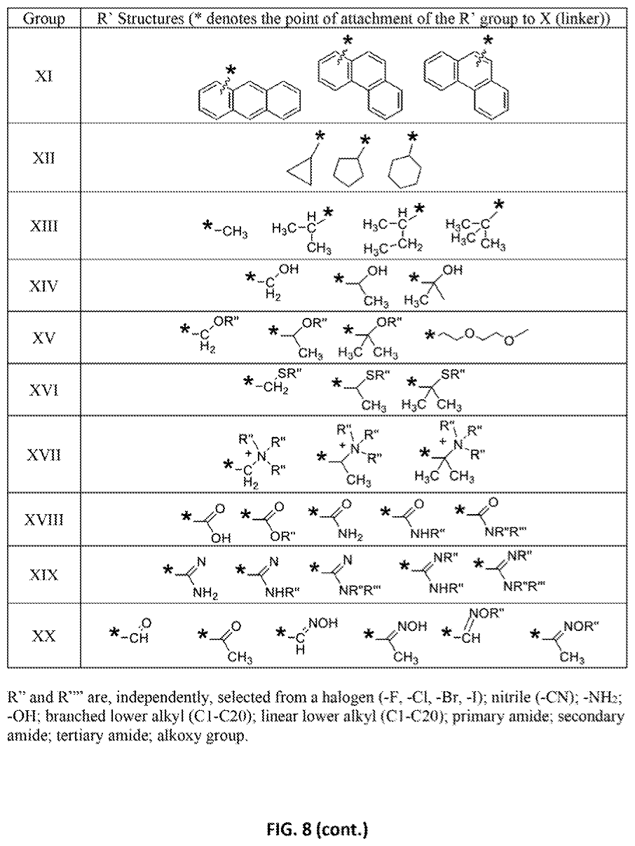

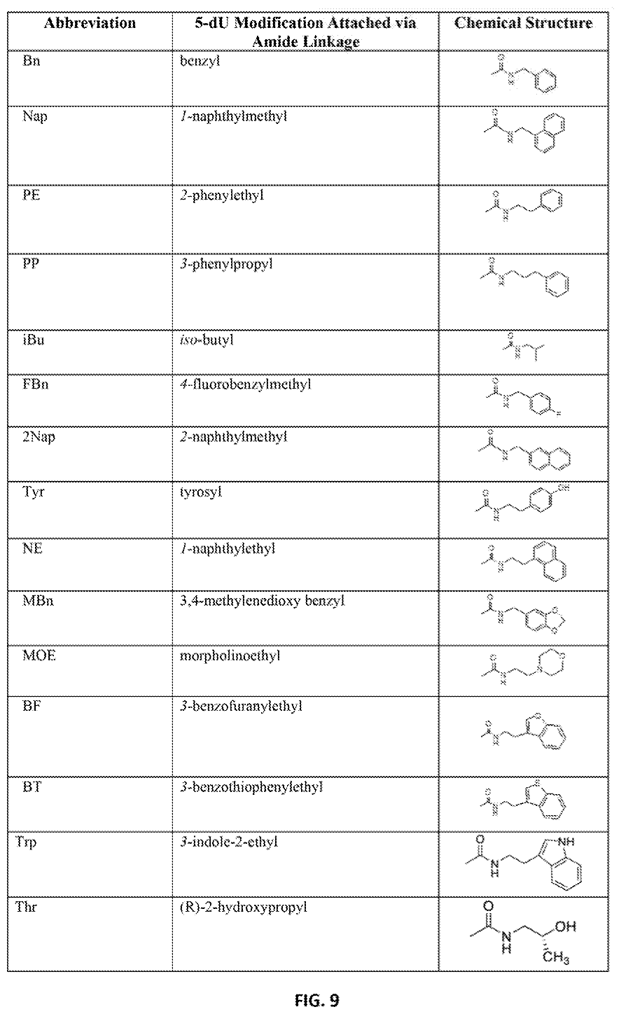

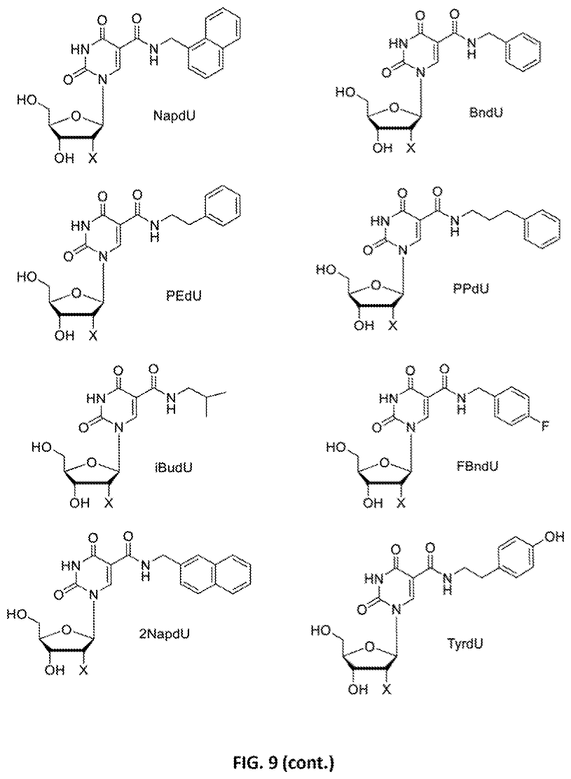

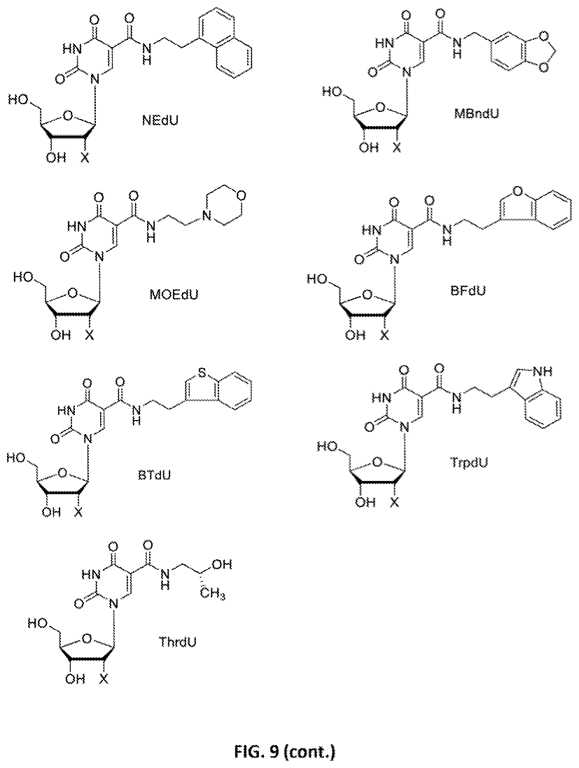

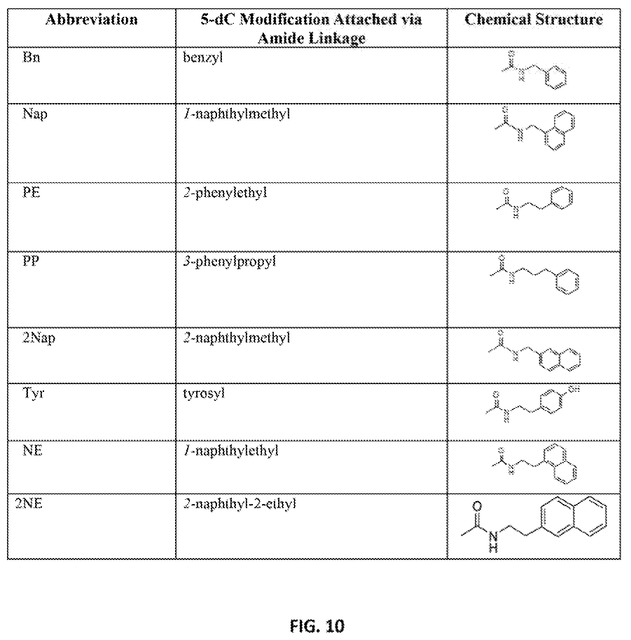

FIG. 7 show the number of analytes in the linear range, above the linear range and below the linear range. The X-axis represents the total protein concentration of the sample, which ranged from about 0 μg/mL to about 50 μg/mL, and the Y-axis for FIG. 7A is the number of analytes measured, and for FIG. 7B, the same data was plotted; however, the Y-axis shows the percent of analytes. Generally, the data of FIG. 7 indicates that a majority of the analytes remained in the linear range when the samples were standardized to a total protein concentration of about 10 μg/mL to just under 50 μg/mL and subject to buffer exchange with assay buffer prior to measuring the analytes with an aptamer-based assay.

Taken together, the data described herein also show that test samples with total protein concentrations significantly greater than about 100 μg/mL, such as that described in Russell et al. “Potential of High-Affinity, Slow Off-Rate Modified Aptamer (SOMAmer) Reagents for Mycobacterium tuberculosis Proteins as Tools for Infection Models and Diagnostic Application.” J. Clin. Microbiol. 55(10): 3072-3088 (2017), have a reduced number of analytes in the linear range than test samples with total protein concentrations at or below 60 μg/mL.

### Example 6: Use of a BCA Assay to Measure Total Protein of Urine Samples

The Micro BCA′ Protein Assay Kit (ThermoFisher; Catalog #23235) was used to measure protein concentrations of urine specimens. The assay was executed in a semi-automated protocol using a Tecan Fluent 780 liquid handling platform. Bovine Serum Albumin (BSA) was used to prepare the protein standards and quality control (QC) samples were commercially purchased from Millipore Sigma (Cat No. NIST927E).

Sixty mL of the Micro BCA Working Reagent were prepared using 30 mL of Reagent A, 28.8 mL of Reagent B, and 1.2 mL of Reagent C. The protein standards and QC samples were prepared in triplicate tubes by diluting a known concentration ranging from 4 to 64 μg/mL with 0.05× Assay Buffer with a final volume of 500 μL per tube. The urine samples were pH-adjusted and underwent buffer exchange, then diluted 20-fold in deionized water.

The protein standards and QC samples were analyzed in triplicate, and “blank” buffer samples were analyzed in duplicate. Urine samples of unknown protein concentration were analyzed as single measurements, but each sample was tested at four different dilutions.

An operator began the Tecan Fluent liquid handling protocol, which included performing several sample dilution steps and mixing the Micro BCA Working Reagent 1:1 with the diluted samples. After completion of the automated protocol, the operator wrapped the samples mixed with working reagent in foil and placed them into a 37° C. incubator for two hours. After the two-hour incubation was complete, an operator centrifuged the samples at 1,000×g for one minute, then measured the absorbance of all samples, including the standards.

A quadratic fit with no weighting was used to create a calibration curve that correlated the absorbance of the standards with their known concentrations. The calibration curve was used to calculate the protein concentrations of the QC samples and the urine samples. In order to verify the accuracy of the results, the concentrations of the duplicate QC samples were calculated and confirmed to be within 20% of their known values.

### Example 7: Use of a BCA Assay in Combination with an Aptamer-Based Assay

As described in Example 3, the total protein concentration in a urine sample affects the number of analytes that are in the linear range in an aptamer-based proteomic assay.

To determine a preferred concentration or range of concentrations of total protein for urine as measured by a BCA assay, a titration series with ten urine samples was analyzed substantially as described in Example 3, and protein concentrations were measured using a BCA assay, as described in Example 6. The number of aptamers (e.g., SOMAmer reagents) that provided a signal in the estimated linear range were counted for each total protein concentration value that was measured.

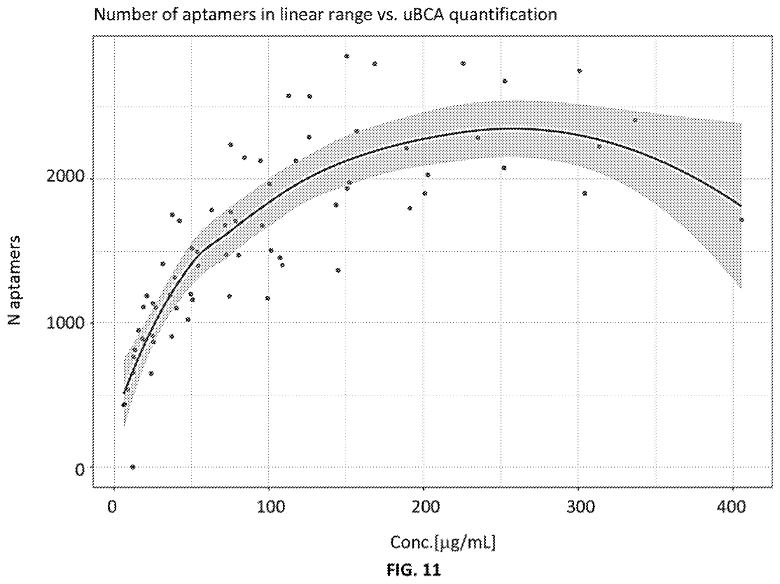

FIG. 11 shows the results, plotted as the number aptamers (e.g., SOMAmer reagents) in the linear range versus the protein concentration as measured by the micro BCA assay described in Example 6. The optimal protein concentration for urine test samples is the lowest concentration at which there does not seem to be further gains in linearity counts. According to the results shown in FIG. 11, that point seems to be at 100 μg/mL or even higher. However, concentrations over 100 μg/mL may not be practical to obtain across variable samples that are typically at a lower concentration. Therefore, the optimal range was determined to be 70-100 μg/mL for the adjusted test samples.

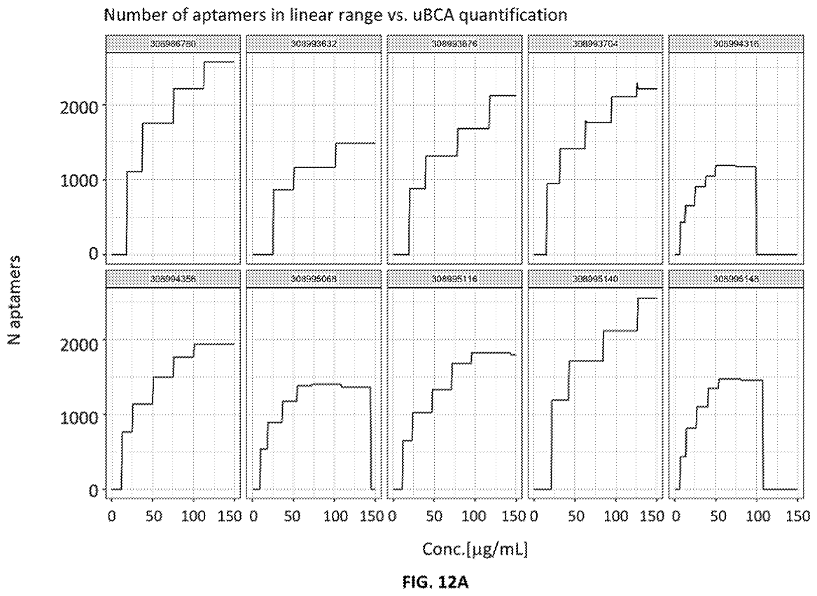

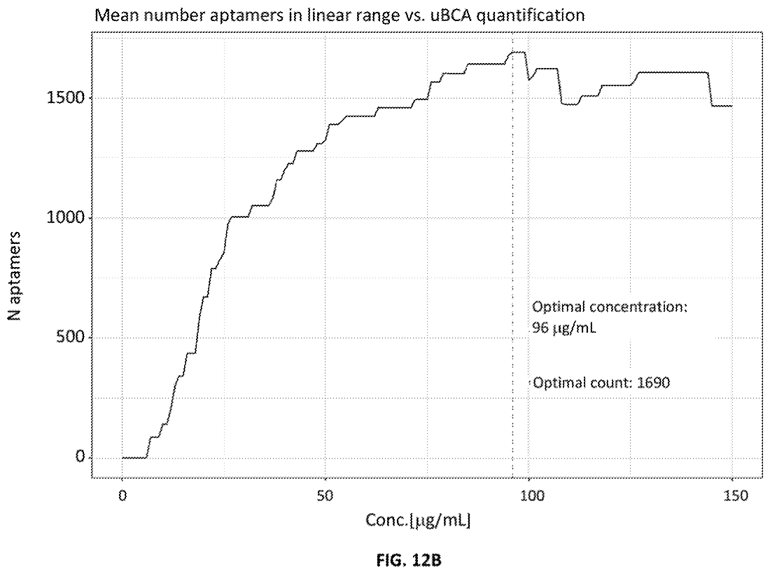

An alternative way of looking at the same data is shown in FIG. 12. The counts of aptamers in the linear range were determined for a range of protein concentrations, between 1 and 150 μg/mL. FIG. 12A shows plots of the counts as functions of the protein concentration, for each of the ten samples tested. FIG. 12B shows the means of these counts, across all ten samples, as a function of protein concentration. As shown in FIG. 12B, the maximum mean counts were achieved at 96 μg/mL; however, the increases in the number of aptamers in the linear range from about 70-85 μg/mL protein concentration and up are small.

Collectively, these data indicate that while a linear range may be obtained for many analytes at total protein concentrations ranging from about 2 μg/mL to about 200 μg/mL, the preferred total protein concentration range that provides for the greatest number of analytes in the linear range along with relatively “strong” RFU signals, was from about 70 μg/mL to 100 μg/mL as measured by micro BCA.

1. A method for preparing a plurality of biological samples for detecting a protein comprising:
generating a plurality of test samples by performing a buffer exchange on the plurality of biological samples using a formulation comprising a buffering agent, one or more salts, a chelating agent and a nonionic surfactant, and
generating a plurality of adjusted test samples by adjusting the total protein concentrations of a plurality of test samples, wherein the total protein concentration of each adjusted test sample is about the same;
wherein the method does not comprise concentrating the total protein of the plurality of biological samples prior to performing the buffer exchange.

2. A method for preparing a biological sample for detecting a protein comprising:
generating a test sample by performing a buffer exchange on the biological sample using a formulation comprising a buffering agent, one or more salts, a chelating agent and a nonionic surfactant; and
generating an adjusted test sample by adjusting the total protein concentration of the test sample, wherein the total protein concentration in the adjusted test sample is from about 70 μg/mL to about 100 μg/mL.

3. The method of claim 1 or 2 comprising determining the protein concentration of the test sample or plurality of test samples and/or the biological sample or plurality of biological samples.

4. A method for preparing a biological sample for detecting a protein comprising:
generating a test sample by performing a buffer exchange on the biological sample using a formulation comprising a buffering agent, one or more salts, a chelating agent and a nonionic surfactant;
determining the total protein concentration of the test sample; and
generating an adjusted test sample by adjusting the total protein concentration of the test sample, wherein the total protein concentration in the adjusted test sample is from about 70 μg/mL to about 100 μg/mL.

5. The method of any one of claims 2-4, comprising generating a plurality of test samples by performing a buffer exchange on a plurality of biological samples, wherein the total protein concentration of each adjusted test sample is about the same.

6. The method of any one of claims 1-5, comprising generating a plurality of test samples by performing a buffer exchange on a plurality of biological samples, wherein the total protein concentration of each adjusted test sample is the same.

7. A method of preparing a biological sample for detecting a protein comprising:
generating a test sample by performing a buffer exchange on the biological sample using a formulation comprising a buffering agent, one or more salts, a chelating agent and a nonionic surfactant;
determining the protein concentration of the test sample; and
if the total protein concentration of the test sample is not in the range of from about 70 μg/mL to about 100 μg/mL, generating an adjusted test sample by adjusting the total protein concentration of the test sample, wherein the total protein concentration in the adjusted test sample is from about 70 μg/mL to about 100 μg/mL.

8. The method of claim 7, wherein if the total protein concentration of the test sample is not in the range of from about 70 μg/mL to about 100 μg/mL; or from about 70 μg/mL to about 95 μg/mL; or from about 70 μg/mL to about 90 μg/mL; or from about 70 μg/mL to about 85 μg/mL; or from about 70 μg/mL to about 80 μg/mL; or from about 70 μg/mL to about 75 μg/mL, generating an adjusted test sample by adjusting the total protein concentration of the test sample to the range.

9. The method of any one of claims 1-8, wherein the one or more salts are each independently selected from a sodium salt, a potassium salt and a magnesium salt.

10. The method of any one of claims 1-8, wherein the one or more salts comprise a sodium salt, a potassium salt and a magnesium salt.

11. The method of any one of claim 9 or 10, wherein the sodium salt is NaCl, the potassium salt is KCl and the magnesium salt is MgCl₂.

12. The method of claim 11, wherein the NaCl in the formulation is at a concentration of from about 10 mM to about 500 mM, or from about 50 mM to about 250 mM, or from about 100 mM to about 200 mM, or from about 75-125 mM, or about 102 mM.

13. The method of claim 11 or 12, wherein the KCl in the formulation is at a concentration of from about 0.5 mM to about 30 mM, or from about 1 mM to about 20 mM, or from about 2 mM to about 15 mM, or from about 4 mM to about 10 mM, or about 5 mM.

14. The method of any one of claims 11-13, wherein the MgCl₂ in the formulation is at a concentration of from about 0.5 mM to about 30 mM, or from about 1 mM to about 20 mM, or from about 2 mM to about 15 mM, or from about 4 mM to about 10 mM, or about 5 mM.

15. The method of any one of claims 1-14, wherein the buffering agent is selected from HEPES, IVIES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.

16. The method of claim 15, wherein the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 50 mM, or about 40 mM.

17. The method of any one of claims 1-16, wherein the chelating agent is selected from EDTA, EGTA, DTPA, BAPTA, DMPS and ALA.

18. The method of claim 17, wherein the chelating agent in the formulation is at a concentration of about 0.1 mM to about 10 mM, or from about 0.5 mM to about 5 mM, or about 1 mM.

19. The method of any one of claims 1-18, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).

20. The method of claim 19, wherein the nonionic surfactant is from about 0.01% to about 1% of the formulation, or about 0.02% to about 0.5% of the formulation, or from about 0.03% to about 0.1% of the formulation, or from about 0.04% to about 0.08% of the formulation, or about 0.05%, or about 0.1% of the formulation, volume for volume.

21. The method of any one of claims 1-20, wherein the total protein concentration of the adjusted test sample is from about 2 μg/mL to about 70 μg/mL; or from about 2 μg/mL to about 75 μg/mL; or from about 4 μg/mL to about 70 μg/mL; or from about 8 μg/mL to about 70 μg/mL; or from about 10 μg/mL to about 70 μg/mL; or from about 10 μg/mL to about 70.

22. The method of any one of claims 3-21, wherein the total protein concentration of the test sample or the adjusted test sample is determined with a bicinchoninic acid (BCA) assay, wherein the BCA assay is optionally a micro BCA assay.

23. The method of claim 22, wherein the assay comprises a bicinchoninic acid reagent or a bicinchonic acid reagent, and optionally an alkaline tartrate-carbonate buffer and/or a copper su fate solution.

24. The method of any one of claims 1-23, wherein the formulation comprises 40 mM HEPES, 102 mM NaCl, 5 mM KCl, 5 mM MgCl₂, 1 mM EDTA and 0.05% Tween-20.

25. The method of claim any one of claims 1-24, wherein the pH of the formulation is from about pH 5 to about pH 9, or from about pH 6 to about pH 8, or from about pH 7 to about pH 7.9, or about pH 7.5.

26. The method of any one of claims 1-25, wherein the formulation consists of 40 mM HEPES, 102 mM NaCl, 5 mM KCl, 5 mM MgCl₂, 1 mM EDTA and 0.05% Tween-20 and has a pH of 7.5.

27. The method of any one of claims 1-26, wherein the buffer exchange is not performed with ultrafiltration.

28. The method of any one of claims 1-27, wherein the buffer exchange is performed with gel filtration chromatography.

29. The method of any one of claims 1-28, wherein the biological sample is a urine sample.

30. The method of any one of claims 1-28, wherein the biological sample is a serum sample.

31. The method of any one of claims 2-30, wherein the method does not comprise a concentrating the biological sample.

32. A test sample comprising a buffer-exchanged biological sample, wherein the test sample has a total protein concentration from about 70 μg/mL to about 100 μg/mL, and wherein the buffer-exchanged biological sample comprises a buffering agent, one or more salts, a chelating agent and a nonionic surfactant.

33. The test sample of claim 32, further comprising one or more protein capture reagents.

34. The test sample of claim 33, wherein each of the one or more protein capture reagents is an aptamer or an antibody.

35. The test sample of any one of claims 32-34, wherein the total protein concentration of the test sample is from about 70 μg/mL to 95 μg/mL; or from about 70 μg/mL to 90 μg/mL; or from about 70 μg/mL to 85 μg/mL; or from about 70 μg/mL to 80 μg/mL; or from about 70 μg/mL to 75 μg/mL; or is about 70 μg/mL.

36. The test sample of any one of claims 32-35, wherein the one or more salts are each independently selected from a sodium salt, a potassium salt and a magnesium salt.

37. The test sample of any one of claims 32-36, wherein the one or more salts comprise a sodium salt, a potassium salt and a magnesium salt.

38. The test sample of claim 36 or 37, wherein the sodium salt is NaCl, the potassium salt is KCl and the magnesium salt is MgCl₂.

39. The test sample of claim 38, wherein the NaCl in the test sample is at a concentration of from about 10 mM to about 500 mM, or from about 50 mM to about 250 mM, or from about 100 mM to about 200 mM or about 102 mM.

40. The test sample of claim 38 or 39, wherein the KCl in the test sample is at a concentration of from about 0.5 mM to about 30 mM, or from about 1 mM to about 20 mM, or from about 2 mM to about 15 mM, or from about 4 mM to about 10 mM, or about 5 mM.

41. The test sample of any one of claims 38-40, wherein the MgCl₂ in the test sample is at a concentration of from about 0.5 mM to about 30 mM, or from about 1 mM to about 20 mM, or from about 2 mM to about 15 mM, or from about 4 mM to about 10 mM, or about 5 mM.

42. The test sample of any one of claims 32-41, wherein the buffering agent is selected from HEPES, IVIES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.

43. The test sample of any one of claims 32-42, wherein the buffering agent in the test sample is at a concentration of from about 4 mM to about 400 mM, or from about 10 mM to about 300 mM, or from about 20 mM to about 200 mM, or from about 30 mM to about 100 mM, or from 35 mM to about 50 mM or about 40 mM.

44. The test sample of any one of claims 32-43, wherein the chelating agent is selected from EDTA, EGTA, DTPA, BAPTA, DMPS and ALA.

45. The test sample of any one of claims 32-44, wherein the chelating agent in the test sample is at a concentration of about 0.1 mM to about 10 mM, or from about 0.5 mM to about 5 mM, or about 1 mM.

46. The test sample of any one of claims 32-45, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).

47. The test sample of any one of claims 32-46, wherein the nonionic surfactant is from about 0.01% to about 1% of the test sample, or about 0.02% to about 0.5% of the test sample, or from about 0.03% to about 0.1% of the test sample, or from about 0.04% to about 0.08% of the test sample, or about 0.05% of the test sample, volume by volume.

48. The test sample of any one of claims 32-47, wherein the total protein concentration of the test sample is determined with an bicinchoninic acid (BCA) assay, wherein the BCA assay is optionally a micro BCA assay.

49. The test sample of claim 48, wherein the assay comprises a bicinchoninic acid reagent or a bicinchonic acid reagent and, optionally, an alkaline tartrate-carbonate buffer and/or a copper sulfate solution.

50. The test sample of any one of claims 32-49, wherein the test sample comprises 40 mM HEPES, 102 mM NaCl, 5 mM KCl, 5 mM MgCl₂, 1 mM EDTA and 0.05% Tween 20.

51. The test sample of any one of claims 32-50, wherein the pH of the test sample is from about pH 5 to about pH 9, or from about pH 6 to about pH 8, or from about pH 7 to about pH 7.9, or about pH 7.5.

52. The test sample of any one of claims 32-51, wherein at least one capture reagent is an aptamer, and wherein the at least one aptamer comprises a 5-position modified pyrimidine.

53. The test sample of claim 52, wherein the 5-position modified pyrimidine comprises a hydrophobic moiety at the 5-position of the pyrimidine.

54. The test sample of claim 53, wherein the hydrophobic moiety is selected from a naphthyl moiety, a phenyl moiety, a tyrosyl moiety, an indole moiety and a morpholino moiety.

55. The test sample of claim 53 or 54, wherein the hydrophobic moiety is covalently linked to the 5-position of the based via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.

56. The test sample of any one of claims 32-55, wherein the biological sample is a urine sample.

57. The test sample of any one of claims 32-55, wherein the biological sample is a serum sample.

58. The test sample of any one of claims 32-57, wherein gel filtration chromatography was used to perform the buffer exchange on the biological sample in order to generate the buffer-exchanged biological sample.

59. A method for detecting a target protein in a test sample comprising:
a) contacting the test sample with at least one capture reagent, wherein the at least one capture reagent is capable of binding to the target protein to form a complex;
b) incubating the test sample with the at least one capture reagent under conditions that allow for the complex to form; and
c) determining the level of the target protein in the test sample by measuring the level of the at least one capture reagent, the complex, or the protein; wherein the level of the at least one capture reagent or the complex is a surrogate for the level of the target protein;
wherein, the test sample is generated by performing a buffer exchange of a biological sample with a formulation comprising a buffering agent, a salt, a chelating agent and a nonionic surfactant; and
wherein, the total protein concentration of the test sample is about 70 μg/mL to about less than 100 μg/mL.

60. The method of claim 59, wherein the at least one protein capture reagent is an aptamer or an antibody.

61. The method of claim 59 or 60 comprising a plurality of capture reagents, where each capture reagent is an aptamer.

62. The method of any one of claims 59-61, wherein the total protein concentration of the test sample is about 70 μg/mL to 95 μg/mL; or about 70 μg/mL to 90 μg/mL; or about 70 μg/mL to 85 μg/mL; or about 70 μg/mL to 80 μg/mL; or about 70 μg/mL to 75 μg/mL or about 70 μg/mL.

63. The method of any one of claims 59-62, wherein the one or more salts are selected from a sodium salt, a potassium salt and a magnesium salt.

64. The method of any one of claims 59-62, wherein the one or more salts comprise a sodium salt, a potassium salt and a magnesium salt.

65. The method of claim 63 or 64, wherein the sodium salt is NaCl, the potassium salt is KCl and the magnesium salt is MgCl₂.

66. The method of claim 65, wherein the NaCl in the formulation is at a concentration of from about 10 mM to about 500 mM, or from about 50 mM to about 250 mM, or from about 100 mM to about 200 mM or about 102 mM.

67. The method of claim 65 or 66, wherein the KCl in the formulation is at a concentration of from about 0.5 mM to about 30 mM, or from about 1 mM to about 20 mM, or from about 2 mM to about 15 mM, or from about 4 mM to about 10 mM, or about 5 mM.

68. The method of any one of claims 65-67, wherein the MgCl₂ in the formulation is at a concentration of from about 0.5 mM to about 30 mM, or from about 1 mM to about 20 mM, or from about 2 mM to about 15 mM, or from about 4 mM to about 10 mM, or about 5 mM.

69. The method of any one of claims 59-68, wherein the buffering agent is selected from HEPES, IVIES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS.

70. The method of any one of claims 59-69, wherein the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 50 mM or about 40 mM.

71. The method of any one of claims 59-70, wherein the chelating agent is selected from EDTA, EGTA, DTPA, BAPTA, DMPS and ALA.

72. The method of any one of claims 59-71, wherein the chelating agent in the formulation is at a concentration of about 0.1 mM to about 10 mM, or from about 0.5 mM to about 5 mM, or about 1 mM.

73. The method of any one of claims 59-72, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80).

74. The method of any one of claims 59-73, wherein the nonionic surfactant is from about 0.01% to about 1% of the formulation, or about 0.02% to about 0.5% of the formulation, or from about 0.03% to about 0.1% of the formulation, or from about 0.04% to about 0.08% of the formulation, or about 0.05% of the formulation.

75. The method of any one of claims 59-74, wherein the total protein concentration of the test sample is measured with a bicinchoninic acid (BCA) assay, wherein the BCA assay is optionally a micro BCA assay.

76. The method of claim 75, wherein the assay comprises a bicinchoninic acid reagent or a bicinchonic acid reagent and, optionally, an alkaline tartrate-carbonate buffer and/or a copper sulfate solution.

77. The method of any one of claims 59-76, wherein the formulation comprises 40 mM HEPES, 102 mM NaCl, 5 mM KCl, 5 mM MgCl₂, 1 mM EDTA and 0.05% Tween 20.

78. The method of any one of claims 59-76, wherein the formulation consists of 40 mM HEPES, 102 mM NaCl, 5 mM KCl, 5 mM MgCl₂, 1 mM EDTA and 0.05% Tween 20 at pH 7.5.

79. The method of any one of claims 59-78, wherein the pH of the formulation is from about pH 5 to about pH 9, or from about pH 6 to about pH 8, or from about pH 7 to about pH 7.9, or about pH 7.5.

80. The method of any one of claims 59-79, wherein the aptamer comprises a 5-position modified pyrimidine.

81. The method of claim 80, wherein the 5-position modified pyrimidine comprises a hydrophobic moiety at the 5-position of the pyrimidine.

82. The method of claim 81, wherein the hydrophobic moiety is selected from a naphthyl moiety, a phenyl moiety, a tyrosyl moiety, an indole moiety and a morpholino moiety.

83. The method of claim 81 or 82, wherein the hydrophobic moiety is covalently linked to the 5-position of the based via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker.

84. The method of any one of claims 59-83, wherein the complex is a non-covalent complex.

85. The method of any one of claims 59-84, wherein the at least one capture reagent is attached to a first solid support before contacting the test sample or the capture reagent is attached to a first solid support after contacting the test sample.

86. The method of any one of claims 59-85, further comprising attaching the complex to a first solid support via the capture reagent.

87. The method of claim 86, further comprising releasing complex from the first solid support and attaching the complex to a second solid support.

88. The method of claim 87, wherein the complex is attached to the second solid support via the protein.

89. The method of any one of claims 59-88, further comprising adding a competitor molecule to the test sample and/or adding a competitor molecule and diluting the test sample.

90. The method of claim 89, wherein the competitor molecule is a polyanionic competitor.

91. The method of claim 90, wherein the polyanionic competitor is selected from an oligonucleotide, polydextran, DNA, heparin and dNTPs.

92. The method of claim 91, wherein the polyanionic competitor is a polydextran, and wherein the polydextran is dextran sulfate.

93. The method of claim 91, wherein the polyanionic competitor is an oligonucleotide, and wherein the oligonucleotide comprises one or more 5-position modified pyrimidines.

94. The method of any one of claims 59-93, wherein the biological sample is urine.

95. The method of any one of claims 59-93, wherein the biological sample is serum.

96. The method of any one of claim 1-31 or 59-86, wherein the buffer exchange is performed using gel filtration chromatography.

97. The method of any one of claim 1-31 or 59-96, comprising measuring the protein concentration of the biological sample prior to the performing of the buffer exchange.

98. The method of claim 97, wherein the protein concentration of the biological sample is measured with an assay selected from the group consisting of a fluorescence readout assay, a bicinchoninic acid (BCA) assay, a micro BCA assay, a Lowry assay, and an ELISA assay.

99. The method of claim 98, wherein the assay is a BCA assay or a micro BCA assay.

100. The method of claim 99, wherein the assay comprises a bicinchoninic acid reagent or a bicinchonic acid reagent and, optionally, an alkaline tartrate-carbonate buffer and/or a copper sulfate solution.

101. The method of claim 98, wherein the assay is a fluorescence readout assay.

102. The method of claim 101, wherein the fluorescence readout assay comprises a reagent selected from a merocyanine dye and epicocconone.

103. A method for preparing a biological sample for detecting a protein comprising:
generating a test sample by performing a buffer exchange on the biological sample using a formulation comprising a buffering agent, one or more salts, a chelating agent and a nonionic surfactant; and
determining the total protein concentration of the biological sample or of the test sample; and
adjusting the total protein concentration of the biological sample or of the test sample, wherein the method does not comprise concentrating the total protein of the biological sample prior to performing the buffer exchange,
optionally wherein (i) the total protein concentration is adjusted before performing the buffer exchange or (ii) the total protein concentration is adjusted after performing the buffer exchange.

104. The method of claim 103, wherein the total protein concentration of the test sample is adjusted after performing the buffer exchange, to from about 2 μg/mL to about 60 μg/mL.

105. The method of claim 103 or 104, comprising generating a plurality of test samples by performing a buffer exchange on a plurality of biological samples, wherein the total protein concentration of each test sample is about the same or is the same.

106. The method of any one of claims 103-105, wherein if the total protein concentration of the biological sample is not in the range of from about 2 μg/mL to about 50 μg/mL; or from about 2 μg/mL to about 45 μg/mL; or from about 4 μg/mL to about 40 μg/mL; or from about 8 μg/mL to about 40 μg/mL; or from about 10 μg/mL to about 40 μg/mL; or from about 10 μg/mL to about 30 μg/mL; or less than 40 μg/mL; or less than 30 μg/mL; or less than 20 μg/mL; or about 15 μg/mL; or from about 10 μg/mL to about 20 μg/mL, the method comprises adjusting the total protein concentration of the biological sample to the range.

107. The method of any one of claims 103-106, wherein the one or more salts are each independently selected from a sodium salt, a potassium salt and a magnesium salt.

108. The method of claim 107, wherein the sodium salt is NaCl, the potassium salt is KCl and the magnesium salt is MgCl₂.

109. The method of any one of claims 103-108, wherein the buffering agent is selected from HEPES, IVIES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS, and wherein the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 50 mM, or about 40 mM.

110. The method of any one of claims 103-109, wherein the chelating agent is selected from EDTA, EGTA, DTPA, BAPTA, DMPS and ALA, and wherein the chelating agent in the formulation is at a concentration of about 0.1 mM to about 10 mM, or from about 0.5 mM to about 5 mM, or about 1 mM.

111. The method of any one of claims 103-110, wherein the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80), and wherein the nonionic surfactant is from about 0.01% to about 1% of the formulation, or about 0.02% to about 0.5% of the formulation, or from about 0.03% to about 0.1% of the formulation, or from about 0.04% to about 0.08% of the formulation, or about 0.05%, or about 0.1% of the formulation, volume for volume.

112. The method of any one of claims 103-111, wherein the total protein concentration of the test sample or the biological sample is determined with an assay selected from the group consisting of a fluorescence readout assay, a Bradford assay, a bicinchoninic acid (BCA) assay, a Lowry assay and an ELISA assay.

113. The method of claim 112, wherein the fluorescence readout assay comprises a reagent selected from a merocyanine dye and epicocconone.

114. The method of any one of claims 103-113, wherein the formulation comprises 40 mM HEPES, 102 mM NaCl, 5 mM KCl, 5 mM MgCl₂, 1 mM EDTA and 0.05% Tween-20, and wherein the pH of the formulation is from about pH 5 to about pH 9, or from about pH 6 to about pH 8, or from about pH 7 to about pH 7.9, or about pH 7.5.

115. The method of any one of claims 103-114, wherein the buffer exchange is performed with gel filtration chromatography.

116. The method of any one of claims 103-115, wherein the biological sample is a urine sample.
